SEQUENCE LISTING (17 sequences)

SEQ ID NO: 1 - protein, 7 aa, Artificial Sequence
  synthetic Ang-(1-7) peptide/analog
DRVYIHP

SEQ ID NO: 2 - protein, 7 aa, Artificial Sequence
  synthetic Ang-(1-7) peptide/analog
  MOD_RES (2)..(2): Xaa is cis-3-(aminomethyl)cyclobutanecarboxylic acid (ACCA)
DXVYIHP

SEQ ID NO: 3 - protein, 7 aa, Artificial Sequence
  synthetic Ang-(1-7) peptide/analog
  MOD_RES (3)..(3): Xaa is cis-3-(aminomethyl)cyclobutanecarboxylic acid (ACCA)
DRXYIHP

SEQ ID NO: 4 - protein, 7 aa, Artificial Sequence
  synthetic Ang-(1-7) peptide/analog
  MOD_RES (4)..(4): Xaa is cis-3-(aminomethyl)cyclobutanecarboxylic acid (ACCA)
DRVXIHP

SEQ ID NO: 5 - protein, 7 aa, Artificial Sequence
  synthetic Ang-(1-7) peptide/analog
  MOD_RES (5)..(5): Xaa is cis-3-(aminomethyl)cyclobutanecarboxylic acid (ACCA)
DRVYXHP

SEQ ID NO: 6 - protein, 7 aa, Artificial Sequence
  synthetic Ang-(1-7) peptide/analog
  MOD_RES (6)..(6): Xaa is cis-3-(aminomethyl)cyclobutanecarboxylic acid (ACCA)
DRVYIXP

SEQ ID NO: 7 - protein, 7 aa, Artificial Sequence
  synthetic Ang-(1-7) peptide/analog
  MOD_RES (2)..(3): Xaa is cis-3-(aminomethyl)cyclobutanecarboxylic acid (ACCA)
DXXYIHP

SEQ ID NO: 8 - protein, 7 aa, Artificial Sequence
  synthetic Ang-(1-7) peptide/analog
  MOD_RES (2)..(2): Xaa is cis-3-(aminomethyl)cyclobutanecarboxylic acid (ACCA)
  MOD_RES (4)..(4): Xaa is cis-3-(aminomethyl)cyclobutanecarboxylic acid (ACCA)
DXVXIHP

SEQ ID NO: 9 - protein, 7 aa, Artificial Sequence
  synthetic Ang-(1-7) peptide/analog
  MOD_RES (2)..(2): Xaa is cis-3-(aminomethyl)cyclobutanecarboxylic acid (ACCA)
  MOD_RES (5)..(5): Xaa is cis-3-(aminomethyl)cyclobutanecarboxylic acid (ACCA)
DXVYXHP

SEQ ID NO: 10 - protein, 7 aa, Artificial Sequence
  synthetic Ang-(1-7) peptide/analog
  MOD_RES (2)..(2): Xaa is cis-3-(aminomethyl)cyclobutanecarboxylic acid (ACCA)
  MOD_RES (6)..(6): Xaa is cis-3-(aminomethyl)cyclobutanecarboxylic acid (ACCA)
DXVYIXP

SEQ ID NO: 11 - protein, 7 aa, Artificial Sequence
  synthetic Ang-(1-7) peptide/analog
  MOD_RES (3)..(4): Xaa is cis-3-(aminomethyl)cyclobutanecarboxylic acid (ACCA)
DRXXIHP

SEQ ID NO: 12 - protein, 7 aa, Artificial Sequence
  synthetic Ang-(1-7) peptide/analog
  MOD_RES (3)..(3): Xaa is cis-3-(aminomethyl)cyclobutanecarboxylic acid (ACCA)
  MOD_RES (5)..(5): Xaa is cis-3-(aminomethyl)cyclobutanecarboxylic acid (ACCA)
DRXYXHP

SEQ ID NO: 13 - protein, 7 aa, Artificial Sequence
  synthetic Ang-(1-7) peptide/analog
  MOD_RES (3)..(3): Xaa is cis-3-(aminomethyl)cyclobutanecarboxylic acid (ACCA)
  MOD_RES (6)..(6): Xaa is cis-3-(aminomethyl)cyclobutanecarboxylic acid (ACCA)
DRXYIXP

SEQ ID NO: 14 - protein, 7 aa, Artificial Sequence
  synthetic Ang-(1-7) peptide/analog
  MOD_RES (4)..(5): Xaa is cis-3-(aminomethyl)cyclobutanecarboxylic acid (ACCA)
DRVXXHP

SEQ ID NO: 15 - protein, 7 aa, Artificial Sequence
  synthetic Ang-(1-7) peptide/analog
  MOD_RES (4)..(4): Xaa is cis-3-(aminomethyl)cyclobutanecarboxylic acid (ACCA)
  MOD_RES (6)..(6): Xaa is cis-3-(aminomethyl)cyclobutanecarboxylic acid (ACCA)
DRVXIXP

SEQ ID NO: 16 - protein, 7 aa, Artificial Sequence
  synthetic Ang-(1-7) peptide/analog
  MOD_RES (5)..(6): Xaa is cis-3-(aminomethyl)cyclobutanecarboxylic acid (ACCA)
DRVYXXP

SEQ ID NO: 17 - protein, 11 aa, Artificial Sequence
  synthetic Ang-(1-7) peptide/analog
  MOD_RES (1)..(1): Xaa may be absent or present; if present, Xaa is norleucine (Nle), Leu, Ala, norvaline (Nva), azidohomoalanine (Aha), or 2-Aminobutyric acid (Abu)
  MISC_FEATURE (1)..(9): may be cyclic peptide with position 1 Xaa and position 9 Xaa joined; may be cyclic peptide with position 2 Xaa and position 9 Xaa joined
  MISC_FEATURE (1)..(9): when cyclized peptide - lactam bridge between Xaa at position 1 and Xaa at position 9 or between Xaa at position 2 and Xaa at position 9
  MOD_RES (2)..(2): Xaa is Asp, N-methyl aspartic acid (NMeD), Ala or N-methyl alanaine (NMeA)
  MOD_RES (3)..(3): Xaa is Arg, N-methyl arginine (NMeR) or cis-3-(aminomethyl)cyclobutanecarboxylic acid (ACCA)
  MOD_RES (4)..(4): Xaa is Val, N-methyl valine (NMeV), Ala, N-methyl alanine (NMeA) or cis-3-(aminomethyl) cyclobutanecarboxylic acid (ACCA)
  MOD_RES (5)..(5): Xaa is Tyr, N-methyl tyrosine (NMeY), Phe, N-methyl phenylalanine (NMeF), Ala, N-methyl alanine (NMeA) or cis-3-(aminomethyl)cyclobutanecarboxylic acid (ACCA)
  MOD_RES (6)..(6): Xaa is Ile, N-methyl isoleucine (NMeI), Ala, N-methyl alanine (NMeA), Leu, N-methyl leucine (NMeL) or cis-3-(aminomethyl)cyclobutanecarboxylic acid (ACCA)
  MOD_RES (7)..(7): Xaa is His, N-methyl histidine (NMeH), Ala, N-methyl alanine (NMeA) or cis-3-(aminomethyl) cyclobutanecarboxylic acid (ACCA)
  MOD_RES (8)..(8): Xaa is Pro, N-methyl proline (NMeP), Ala or N-methyl alanine (NMeA)
  MOD_RES (9)..(9): Xaa is Lys, ornithine (Orn), 2,3- diaminopropionic acid (Dap), 2,4-diaminobutyric acid (Dab) or N-methyl lysine (NMe-K); Xaa may be amidated if residue 10 absent
  MOD_RES (10)..(11): Xaa may be absent, present as a single amino acid (D-valine (dV) or D-proline (dP)) or present as two amino acids (D-valine-D-proline (dV-dP))
XXXXXXXXXXX

(19) United States
(12) Patent Application Publication
(10) Pub. No.: US 2021/0024578 A1
(43) Pub. Date: Jan. 28, 2021

(54) ANGIOTENSIN-(1-7) ANALOGS AND METHODS RELATING THERETO

(71) Applicants: WAKE FOREST UNIVERSITY HEALTH SCIENCES, Winston-Salem, NC (US); UNIVERSITY COLLEGE DUBLIN, Dublin (IE)

(72) Inventors: Patricia E. Gallagher, Lewisville, NC (US); E. Ann Tallant, Lewisville, NC (US); Francesca Paradisi, Nottingham (GB); Mark C. Chappell, Winston-Salem, NC (US)

(21) Appl. No.: 16/918,907

(22) Filed: Jul. 1, 2020

Related U.S. Application Data
(63) Continuation of application No. 15/779,598, filed on May 29, 2018.
(60) Provisional application No. 62/266,410, filed on Dec. 11, 2015.

(30) Foreign Application Priority Data
Dec. 11, 2015 (CA) 2914601

(51) Int. Cl. C07K 7/14; A61P 35/00

(52) U.S. Cl. CPC C07K 7/14; A61K 38/00; A61P 35/00

(57) ABSTRACT

Angiotensin (1-7) analogs are provided. The analogs contain one or more substitutions with non-natural amino acid cis-3-(aminomethyl)cyclobutanecarboxylic acid (ACCA). Also provided are methods of making such analogs and methods for using such analogs as therapeutic compositions to treat or prevent various diseases or conditions.

### CROSS-REFERENCE TO RELATED APPLICATIONS

This application is a continuation of U.S. Nonprovisional application Ser. No. 15/779,598, filed May 29, 2018, which is a 371 national stage of International Application No. PCT/US2016/066181, filed Dec. 12, 2016, which claims the benefit of priority to U.S. Provisional Application No. 62/266,410, filed Dec. 11, 2015, and Canadian Application No. 2,914,601, filed Dec. 11, 2015, which are each incorporated by reference herein in their entirety.

### REFERENCE TO A SEQUENCE LISTING SUBMITTED AS A TEXT FILE VIA EFS-WEB

The official copy of the sequence listing is submitted electronically via EFS-Web as an ASCII formatted sequence listing with a file named 1202065_SequenceListing, created on Oct. 6, 2020, and having a size of 8,513 bytes. The sequence listing contained in this ASCII formatted document is part of the specification and is herein incorporated by reference in its entirety.

### BACKGROUND

Angiotensin-(1-7) (“Ang-(1-7)”) is an endogenous, seven amino acid peptide hormone having the sequence Asp¹-Arg²-Val³-Tyr⁴-Ile⁵-His⁶-Pro⁷ (SEQ ID NO:1). Ang-(1-7) mediates biological responses by activating mas, a unique G protein-coupled receptor, thereby providing specific targeted actions when used as a therapeutic (Santos et al., Proc. Natl. Acad. Sci. USA 100:8258-8263 (2003) and George et al., Nat. Rev. Cancer 10:745-759 (2010)). Ang-(1-7) is primarily derived from angiotensin I (Ang I) by tissue peptidases, including neprilysin, thimet oligopeptidase and prolyl endopeptidase (Ferrario et al., Hypertension 30:535-541 (1997)), and by angiotensin converting enzyme (ACE) 2 from angiotensin II (Ang II) (Vickers et al., J. Biol. Chem. 277:14836-14843 (2002)), Ang-(1-7) is a substrate for angiotensin converting enzyme (ACE), which hydrolyzes the bond between isoleucine and histidine at positions 5 and 6 of the peptide to yield the dipeptide His-Pro and the pentapeptide Ang(1-5) but does not further cleave this fragment (Chappell et al. Hypertension 31:362-367 (1998)). As a result, the half-life of Ang (1-7) in vivo is short. Ang (1-7) inhibits the growth of human lung cancer (Gallagher and Tallant, Carcinogenesis 25:2015-52 (2003), Menon et al., Cancer Res. 15:2809-15 (2007), and Soto-Pantoja et al., Mol. Canc. Ther. 8:1676-83 (2009)), breast cancer (Cook et al., Cancer Res. 70:8319-28 (2010)) and prostate cancer (Krishnan et al., The Prostate 73:60-70 (2013) and Krishnan et al., The Prostate 73:71-82 (2013). However, the half-life in human patients administered the heptapeptide hormone for the treatment of cancer was between 25 and 37 min (Petty et al., Clinical Cancer Research 15:7398-404 (2009)) in agreement with previous studies in breast cancer patients treated with Ang-(1-7) as adjuvant therapy (Rodgers et al., Cancer Chemother. Pharmacol. 57:559-68 (2006)).

### BRIEF SUMMARY

Described herein are Ang-(1-7) peptide analogs. In some instances, they may used as agonists for the angiotensin (1-7) receptor mas. Also provided herein are methods for use of the peptide analogs in treating cancer in a subject. The class of Ang-(1-7) peptide analogs described herein includes peptides of the following formula X¹—X²—X³—X⁴—X⁵—X⁶—X⁷ and pharmaceutically acceptable salts thereof. The peptide analogs have sequence similarity to Ang-(1-7). In particular, the peptide analogs include at least one cis-3-(aminomethyl)cyclobutanecarboxylic acid (ACCA), a non-natural δ-amino acid. In these peptide analogs, X¹ may be aspartic acid, N-methyl aspartic acid, alanine, or N-methyl alanine; X² may be arginine, N-methyl arginine, or ACCA; X³ may be valine, N-methyl valine, alanine, N-methyl alanine, or ACCA; X⁴ may be tyrosine, N-methyl tyrosine, phenylalanine, N-methyl phenylalanine, alanine, N-methyl alanine, or ACCA; X⁵ may be isoleucine, N-methyl isoleucine, alanine, N-methyl alanine, leucine, N-methyl leucine, or ACCA; X⁶ may be histidine, N-methyl histidine, alanine, N-methyl alanine, or ACCA; and X⁷ may be proline, N-methyl proline, alanine, or N-methyl alanine. At least one of X², X³, X⁴, X⁵, and X⁶ is ACCA.

Also described herein are pharmaceutical compositions including a compound as described herein and a pharmaceutically acceptable carrier.

Further described herein are methods of treating or preventing various diseases and conditions in a subject. The methods of treating or preventing such diseases and conditions include administering to a subject an effective amount of an angiotensin-(1-7) peptide analog, or pharmaceutical composition comprising such an analog, as described herein.

The details of one or more aspects and embodiments are set forth in the description and drawings below. Other features, objects, and advantages will be apparent from the description and drawings, and from the claims.

### BRIEF DESCRIPTION OF THE DRAWINGS

FIG. 1A shows the chemical structure of an exemplary Ang-(1-7) peptide analog having ACCA substituted at position X⁵ in place of isoleucine according to one aspect. This peptide structure corresponds to SEQ ID NO:5.

FIG. 1B shows the chemical structure of an exemplary Ang-(1-7) peptide analog having ACCA substituted at position X⁶ in place of histidine according to one aspect. This peptide structure corresponds to SEQ ID NO:6.

FIG. 1C shows the chemical structure of an exemplary Ang-(1-7) peptide analog having ACCA substituted at position X³ in place of valine according to one aspect. This peptide structure corresponds to SEQ ID NO:3.

FIGS. 2A-4D show graphs depicting enzymatic digestion by angiotensin converting enzyme (ACE) of Ang-(1-7), Ang-(1-7)-ACCA⁵, Ang-(1-7)-ACCA⁶, and Ang-(1-7)-ACCA³, respectively, in accordance with one aspect. The peptides (100 μM) were incubated with human ACE (1.0 μg) in reaction buffer for 2 hours at 37° C. in a final volume of 100 μL, and the reaction products assessed by HPLC (UV 220 nm).

FIGS. 3A-5D show graphs depicting enzymatic digestion by dipeptidyl peptidase III (DPE III) of Ang-(1-7), Ang-(1-7)-ACCA⁵, Ang-(1-7)-ACCA⁶, and Ang-(1-7)-ACCA³, respectively, in accordance with one aspect. The peptides (100 μM) were incubated with human DPE III (1.0 μg) in reaction buffer for 2 hours at 37° C. in a final volume of 100 μL, and the reaction products assessed by HPLC (UV 220 nm).

FIG. 4A and FIG. 4B show graphs depicting growth inhibition observed in 4T1 murine triple negative breast cancer cells and human HT-1080 sarcoma cells, respectively, that were treated with (i) 100 nM Ang-(1-7) or (ii) Ang-(1-7)-ACCA⁵, Ang-(1-7)-ACCA⁶, or Ang-(1-7) -ACCA³ at 100 nM or 1 μM, in accordance with one aspect. Cells were grown in 24 well cluster plates in DMEM with 1% FBS containing the test compound for 3 days. Ang-(1-7) was added daily. Cell number was counted using a Nexelcom Cellometer. Breast cancer cells n=3 in duplicate; sarcoma cells n=2 in duplicate. * denotes p<0.05; ** denotes p<0.01; *** denotes p <0.001.

### DETAILED DESCRIPTION

Described herein are angiotensin (1-7) [Ang-(1-7)] peptide analogs containing non-natural amino acids and methods for their use. The peptide analogs are resistant to degradation by enzymes that degrade Ang-(1-7) and, thus, are more stable. The analogs also have similar biological activity to Ang-(1-7).

### I. Angiotensin (1-7) Analogs

A class of Ang-(1-7) peptide analogs described herein is represented generally by Formula I: X¹—X²—X³—X⁴—X⁵—X⁶—X⁷ and pharmaceutically acceptable salts thereof. Positions X¹—X⁷ correspond to amino acid positions relative to the amino acids of the Ang-(1-7) peptide. In this class of peptide analogs, certain amino acid positions are substituted with non-natural δ-amino acid cis-3-(aminomethyl)cyclobutanecarboxylic acid (ACCA). ACCA contains a cyclobutane ring that gives the molecule conformational rigidity and locks the amino and carboxylic acid group in a cis conformation. X¹ and X⁶ may be the native amino acid of the Ang-(1-7) peptide (Arg and Pro, respectively) or substituted with a conservative amino acid. At least one of X², X³, X⁴, X⁵, or X⁶ may be substituted with ACCA. In some instances, at least two of X², X³, X⁴, X⁵, or X⁶ may be substituted with ACCA. In some instances, at least one of X¹, X², X³, X⁴, X⁵, X⁶, or X⁷ may be substituted with a conservative amino acid and at least one of X², X³, X⁴, X⁵, or X⁶ may be substituted with ACCA. The analogs incorporating ACCA have decreased enzymatic degradation, thus, overcoming the problem of the short half-life of the Ang-(1-7) heptapeptide. Without being held to any particular theory, inclusion of ACCA at one or more of positions X², X³, X⁴, X⁵, or X⁶ may sufficiently weaken the binding of an enzyme that degrades Ang-(1-7) to the peptide analogs so as to reduce degradation of the peptides.

With respect to Formula I, in some instances, X¹ may be aspartic acid, N-methyl aspartic acid, alanine, or N-methyl alanine. In some instances, X² may be arginine, N-methyl arginine, or ACCA. In some instances, X³ may be valine, N-methyl valine, alanine, N-methyl alanine, or ACCA. In some instances, X⁴ may be tyrosine, N-methyl tyrosine, phenylalanine, N-methyl phenylalanine, alanine, N-methyl alanine, or ACCA. In some instances, X⁵ may be isoleucine, N-methyl isoleucine, alanine, N-methyl alanine, leucine, N-methyl leucine, or ACCA. in some instances, X⁶ may be histidine, N-methyl histidine, alanine, N-methyl alanine, or ACCA. In some instances, X⁷ may be proline, N-methyl proline, alanine, or N-methyl alanine.

In some instances, the peptide analogs may have ACCA at one of X², X³, X⁴, X⁵, or X⁶. In some instances, the peptide analogs may have ACCA at two of X², X³, X⁴, X⁵, or X⁶. In some instances, the peptide analogs may have ACCA at X² and X³, at X² and X⁴, at X² and X⁵, at X² and X⁶, at X³ and X⁴, at X³ and X⁵, at X³ and X⁶, at X⁴ and X⁵, at X⁴ and X⁶, or at X⁵ and X⁶. In some instances, the peptide analogs include peptides having the sequences identified in Table 1 and as set forth in SEQ ID NOs. 2-16. In some instances, peptide analogs containing a single ACCA substitution may have an amino acid sequence as set forth in any one of SEQ ID NOs. 2-6. In some instances, peptide analogs containing a double ACCA substitution may have an amino acid sequence as set forth in any one of SEQ ID NOs. 7-16.

TABLE 1
Ang-(1-7) Peptide and Analogs

| SEQ ID NO | Amino Acid Sequence |
|---|---|
| SEQ ID NO: 1 | Asp-Arg-Val-Tyr-Ile-His-Pro |
| SEQ ID NO: 2 | Asp-ACCA-Val-Tyr-Ile-His-Pro |
| SEQ ID NO: 3 | Asp-Arg-ACCA-Tyr-Ile-His-Pro |
| SEQ ID NO: 4 | Asp-Arg-Val-ACCA-Ile-His-Pro |
| SEQ ID NO: 5 | Asp-Arg-Val-Tyr-ACCA-His-Pro |
| SEQ ID NO: 6 | Asp-Arg-Val-Tyr-Ile-ACCA-Pro |
| SEQ ID NO: 7 | Asp-ACCA-ACCA-Tyr-Ile-His-Pro |
| SEQ ID NO: 8 | Asp-ACCA-Val-ACCA-Ile-His-Pro |
| SEQ ID NO: 9 | Asp-ACCA-Val-Tyr-ACCA-His-Pro |
| SEQ ID NO: 10 | Asp-ACCA-Val-Tyr-Ile-ACCA-Pro |
| SEQ ID NO: 11 | Asp-Arg-ACCA-ACCA-Ile-His-Pro |
| SEQ ID NO: 12 | Asp-Arg-ACCA-Tyr-ACCA-His-Pro |
| SEQ ID NO: 13 | Asp-Arg-ACCA-Tyr-Ile-ACCA-Pro |
| SEQ ID NO: 14 | Asp-Arg-Val-ACCA-ACCA-His-Pro |
| SEQ ID NO: 15 | Asp-Arg-Val-ACCA-Ile-ACCA-Pro |
| SEQ ID NO: 16 | Asp-Arg-Val-Tyr-ACCA-ACCA-Pro |

In some instances, the peptide analogs may include a conservative substitution at any of X¹—X²—X³—X⁴—X⁵—X⁶—X⁷ that is not substituted with ACCA. The peptide analogs may contain 0, 1, or 2 conservative amino acid substitutions at any of X¹—X²—X³—X⁴—X⁵—X⁶—X⁷ that is not substituted with ACCA. In some instances, the peptide analogs of the disclosure have an amino acid sequence substantially similar to any of SEQ ID NOs. 2-17 but containing at least one amino acid substitution. For example, the analogs may comprise amino acid sequences that are at least 80%, at least 70%, or at least 57% identical to the sequences set forth in SEQ ID NOs. 2-17. In some instances, the peptide analogs may include conservative amino acid substitutions, wherein conservative amino acid substitutions are those substitutions which do not significantly affect the structure or function of the peptide analogs. Exemplary conservative amino acid substitutions are described in U.S. Provisional Application No. 62/220,711, filed Sep. 18, 2015, which is incorporated herein by reference in its entirety. In some instances, the peptide analogs may include natural amino acids, non-natural amino acids, or both natural and non-natural amino acids. In some instances, the peptide analogs may include L form amino acids. In some instances, the peptide analogs may include D form amino acids. In some instances, the peptide analogs may include beta form amino acids. In some instances, the peptide analogs may include at least one of L form amino acid, D form amino acid, or beta form amino acid. In some instances, the peptide analogs may contain one or more methylated amino acids.

In some instances, the peptide analogs may contain additional N′ terminal or C′ terminal amino acids. For example, in some instances, the peptide analogs may be modified to contain an additional N′ terminal amino acid (referred to as N¹). In some instances, N¹ may be norleucine (Nle), leucine (L), alanine (A), norvaline (Nva), azidohomoalanine (Aha), or 2-Aminobutyric acid (Abu). In some instance, N¹ may be modified with a —COCH₃ group (acetylated) or modified with a —NH₂ group (aminated). In some instances, the peptide analogs may be modified to contain one or more additional C′ terminal amino acids. Where the peptide analogs contain one or more additional C′ terminal amino acids, the amino acid attached to Pro⁷ (X⁷) is referred to as C¹. In some instances, C¹ may be lysine (K), ornithine (Orn), 2,3-diaminopropionic acid (Dap), 2,4-diaminobutyric acid (Dab), or N-methyl lysine (NMe-K). Optionally, C¹ may be modified by —NH₂ (amidated). In some instances, the analog peptides may have one or more additional amino acids attached to C¹. In some instances, the analogs may include —C¹—Y¹ attached to Pro⁷ (X⁷). Y¹ may be absent, may be a single amino acid, or may be two amino acids. For example, Y¹ may be D-valine-D-proline (dV-dP), D-valine (dV), or D-proline (dP), or may be absent. In some instances, C¹ may be modified with an —NH₂ if Y₁ is absent. In some instances, the peptide analogs may have the formula N¹—X¹—X²—X³—X⁴—X⁵—X⁶—X⁷—C¹—Y¹.

In some instances, the Ang-(1-7) peptide analogs may have a cyclic structure. In some instances, the analogs may be cyclic peptides in which the amino termini and carboxyl termini, amino termini and side chain, carboxyl termini and side chain, or side chain and side chain are linked with a covalent bond that generates a ring. In some instances, cyclization further stabilizes the peptides in vivo. In some instances, the peptide analogs may have increased resistance to degradation due to the digestive process. In some instances, the peptide analogs may be cyclized via a lactam bridge. In some instances, cyclic peptide analogs contain additional amino acid to facilitate cyclization. In one example, the peptide analogs have the formula N¹—X¹—X²—X³—X⁴—X⁵—X⁶—X⁷—C¹—Y¹ and N¹ or X¹ is connected to C¹ via a lactam bridge thereby cyclizing the peptide. In some instances, the peptide analog comprises the sequence set forth in SEQ ID NO:17.

In some instances, the peptide analogs described by Formula I and pharmaceutically acceptable salts thereof, or derivatives thereof, are more stable than native Ang-(1-7) peptide. In some instances, the peptides have a longer half-life under physiological conditions than Ang-(1-7) peptide. Physiological conditions are environments within the body of a subject to which the peptides may be administered. Exemplary physiological environments include blood, plasma, serum, saliva, and the environments of the gastrointestinal tract, the nasal passage, the respiratory tract, and the lungs. For example, Ang-(1-7) has a half-life of 30 minutes in plasma. In some instances, the half-life of the peptides described herein, and pharmaceutically acceptable salts or derivatives thereof, may be at least about 2 times, 3 times, 4 times, 5 times, 6, times, 10 times, 15 times, 20 times, 40 times, 60 times, 80 times, 100 times, 125 times, 150 times, 175 times, or 200 times longer.

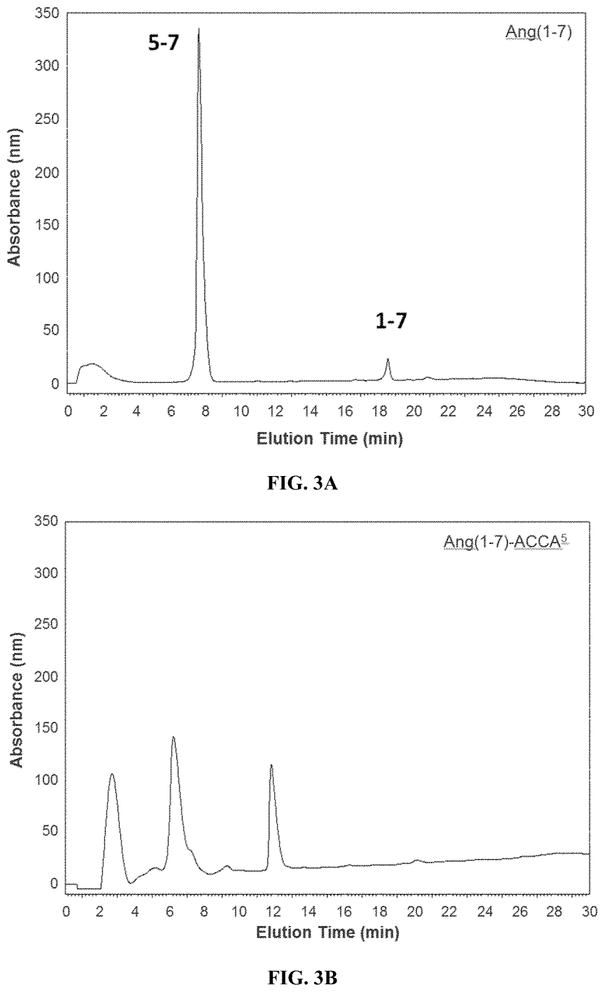

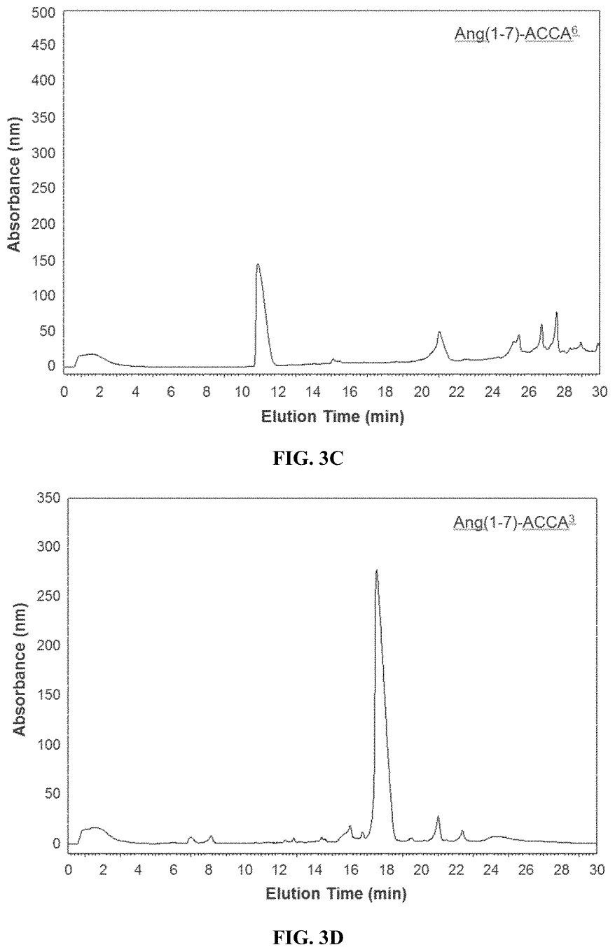

The peptide analogs provided may have similar or improved biological activity compared to Ang-(1-7). In some instances, the analogs may be resistant to degradation by enzymes that degrade Ang-(1-7). In some instances, peptide analogs containing a single ACCA substitution may be resistant to degradation by different types of enzymes or may be resistant to degradation by one type of enzyme. In some instances, peptide analogs containing a double ACCA substitution (that is an ACCA substitution at a first position and an ACCA substitution at a second position) may be resistant to degradation by different types of enzymes where a peptide containing an ACCA substitution at either the first position or the second position may be degraded by one or more of the different types of enzymes. In some instances, peptide analogs containing an ACCA substitution at X³, X⁵, or X⁶ may be resistant to degradation by angiotensin converting enzyme (ACE). For example, as shown in FIGS. 2A-2D, exemplary analogs Ang-(1-7) -ACCA⁵ and Ang-(1-7)-ACCA⁶ may be resistant to degradation by ACE, while Ang-(1-7) was readily degraded. In another example, peptide analogs containing an ACCA substitution at X³ or X⁵ may be resistant to degradation by dipeptidyl peptidase III (DPE III). For example, as shown in FIGS. 3A-3D, exemplary analogs Ang-(1-7)-ACCA⁵ and Ang-(1-7)-ACCA³ were resistant to degradation by DPE III, while Ang-(1-7) was readily degraded. In one example, to be resistant to degradation by ACE and DPE III, a peptide analog having an ACCA substitution at X⁶ may also have an ACCA substitution at one of X², X³, X⁴, or X⁶ or may be cyclized via a lactam bridge as described above.

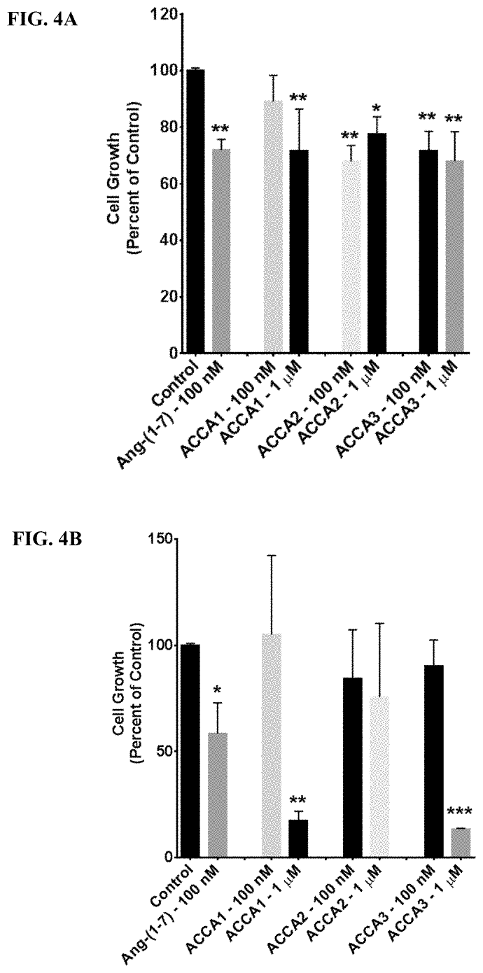

In some instances, the ACCA substituted peptide analogs of this disclosure are resistant to degradation by ACE, DPE III, or both, which may increase stability of the analogs in vivo by preventing key degradation mechanisms. In some instances, the peptide analogs may also have similar biological activity to that of Ang-(1-7) but may be more stable in biological conditions. For example, the analogs may be stable in a biological system, or similar conditions, such as, for example, culture conditions, for at least 3 days. For example, as shown in FIG. 4A and FIG. 4B, exemplary analogs Ang-(1-7)-ACCA⁵ and Ang-(1-7)-ACCA⁶, having ACCA substitutions at X⁵ and X⁶, respectively, were able to reduce proliferation of 4T1 murine triple negative breast cancer cells and HT-1080 human sarcoma cells over the course of 3 days incubation when added at the start of the incubation. In contrast, Ang-(1-7) was added each day to control cells in order to maintain effective inhibition of cell growth over the course of the incubation period. Thus, in some instances, the ACCA substituted peptide analogs effectively inhibit cell proliferation of cancer cells at least three times longer than Ang-(1-7).

### II. Methods of Making the Angiotensin (1-7) Analogs

The peptides described herein can be prepared in a variety of ways. The peptides can be synthesized using various synthetic methods. The peptides described herein can be prepared from readily available starting materials. Optimum reaction conditions can vary with the particular reactants or solvent used, but such conditions can be determined by one skilled in the art by routine optimization procedures.

Variations on Formula I include the addition, subtraction, or movement of the various constituents as described for each compound. Similarly, when one or more chiral centers are present in a molecule, all possible chiral variants are included. Additionally, compound synthesis can involve the protection and deprotection of various chemical groups. The use of protection and deprotection and the selection of appropriate protecting groups can be determined by one skilled in the art. The chemistry of protecting groups can be found, for example, in Wuts, Greene's Protective Groups in Organic Synthesis, 5th. Ed., Wiley & Sons, 2014, which is incorporated herein by reference in its entirety.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, such as temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (such as ¹H or ¹³C), infrared spectroscopy, spectrophotometry (such as UV-visible), or mass spectrometry, or by chromatography such as high performance liquid chromatography (HPLC) or thin layer chromatography.

Peptides described by Formula I and pharmaceutically acceptable salts thereof can be made using fluorenylmethyloxycarbonyl (FMOC) solid phase peptide synthesis. ACCA may be synthesized in seven steps as shown in Example 1 (Scheme 1) and can be FMOC-protected to allow for its incorporation into peptides by solid phase peptide synthesis.

Generally, the amino acids from which the peptide analogs are derived can be naturally occurring amino acid residues, non-natural amino acid residues, or combinations thereof. The twenty common naturally-occurring amino acid residues are as follows: A (Ala, alanine), R (Arg, arginine); N (Asn, asparagine); D (Asp, aspartic acid); C (Cys, cysteine) Q (Gln, glutamine), E (Glu, glutamic acid); G (Gly, glycine); H (His, histidine); I (Ile, isoleucine); L (Leu, leucine); K (Lys, lysine); M (Met, methionine); F (Phe, phenylalanine); P (Pro, proline); S (Ser, serine); T (Thr, threonine); W (Trp, tryptophan); Y (Tyr, tyrosine); and V (Val, valine). The peptides of this disclosure also contain at least one non-natural amino acid residue, cis-3-(aminomethyl)cyclobutanecarboxylic acid (ACCA).

Various peptide synthesis methods and conditions are contemplated. For example, the peptide synthesis can be performed using 1-[Bis(dimethylamino)methylene]-1H-1,2,3-triazolo[4,5-b]pyridinium 3-oxid hexafluorophosphate (HATU) and proline pre-loaded 2CT resin to which FMOC-protected amino acid residues are sequentially added. The amino acid additions may be single coupling cycles. In some instances, the amino acid addition may be a double coupling cycle such as, for example, when coupling to the resin-bound proline. In some instances, HATU/N,N-diisopropylethylamine (DIEA) coupling chemistry in N-methylpyrrolidone (NMP) solvent may be used. In some instances, a 10-fold to 15-fold excess of FMOC-protected amino acid to resin may be used for each coupling reaction.

The nature and use of protecting groups is well known in the art. Generally, a suitable protecting group is any sort of group that can help prevent the atom to which it is attached, typically oxygen or nitrogen, from participating in undesired reactions during processing and synthesis. Protecting groups include side chain protecting groups and amino- or N-terminal protecting groups. Protecting groups can also prevent reaction or bonding of carboxylic acids, thiols, and the like. In some instances, histidine, tyrosine, aspartic acid, and arginine may require side-chain protecting groups during the synthesis of the Ang-(1-7) peptide analogs. For example, tripheylmethyl (Trt) may be used as protecting group for histidine. In another example, tert-butyl (t-Bu) may be used as a protecting group for tyrosine, aspartic acid, or both. In another example, 2,2,4,6,7-pentametyldihydrobenzofuran-5-sulfonyl (Pbf) may be used as a protecting group for arginine.

In some instances, peptide cleavage from the resin and side chain deprotection may be achieved with a deprotecting agent such as, for example, trifluoroacetic acid (TFA) and triisopropylsilane (TIPS). In some instances, such cleavage and deprotection may also include scavenger molecules such as thioanisole.

In some instances, the peptide analogs may be cyclized during peptide synthesis. For example, the Ang-(1-7) peptide analogs may be modified to contain a lactam bridge as described above in Section I. An exemplary method of forming cyclized Ang-(1-7) peptide analogs is described in U.S. Provisional Application No. 62/220,711, filed Sep. 18, 2015, which is incorporated herein by reference in its entirety.

### III. Pharmaceutical Formulations

The peptides described herein or derivatives thereof can be provided in a pharmaceutical composition. Depending on the intended mode of administration, the pharmaceutical composition can be in the form of solid, semi-solid, liquid dosage forms, or combinations thereof, such as, for example, tablets, suppositories, pills, capsules, powders, liquids, or suspensions, preferably in unit dosage form suitable for single administration of a precise dosage. The compositions include a pharmaceutically effective amount of the peptides described herein, or derivatives thereof, in combination with a pharmaceutically acceptable carrier and, in addition, may include other medicinal agents, pharmaceutical agents, carriers, or diluents. The term pharmaceutically acceptable refers to a material that is not biologically or otherwise undesirable, which can be administered to an individual along with the selected compound without causing unacceptable biological effects or interacting in a deleterious manner with the other components of the pharmaceutical composition in which it is contained.

In some instances, the concentration of the peptide in a liquid pharmaceutical formulation may be in the range of about 10 mg/ml to about 200 mg/ml, or about 25 mg/ml to about 175 mg/ml, or about 40-70 mg/ml, or about 40 to about 60 mg/ml, or ranges therein. For example, the formulation may have a concentration of about 20 mg/ml, 30 mg/ml, 40 mg/ml, 50 mg/ml, 60 mg/ml, 70 mg/ml, 80 mg/ml, 90 mg/ml, or 100 mg/ml. In some instances, the concentration of the peptide may be up to 50 mg/ml, up to 100 mg/ml, up to 150 mg/ml, or up to 200 mg/ml. In some instances, the amount of the peptide in a solid pharmaceutical formulation may be in the range of about 5 mg to 1 gram, or about 10 mg to 60 mg, or about 25 mg to 75 mg, or about 50 to 150 mg, or about 75 mg to 200 mg, or about 150 mg to 300 mg, or about 250 mg to 500 mg, or about 350 mg to 650 mg, or about 500 mg to 750 mg, or about 10 mg to 500 mg, or about 100 mg to 500 mg, or about 400 mg to 750 mg. For example, the formulation may have an amount of peptide of 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1 gram. In some instances, the amount of the peptide may be up to 100 mg, 150 mg, 200 mg, 250 mg, 300 mg, 350 mg, 400 mg, 450 mg, 500 mg, 550 mg, 600 mg, 650 mg, 700 mg, 800 mg, 900 mg, or 1000 mg. In some instances, the amount of the peptide in a semi-solid pharmaceutical formulation may be in the range of about 0.1% to 50%, or about 1% to 10%, or about 5% to 15%, or about 10% to 20%, or about 15% to 25%, or about 20% to 30%, or about 25% to 35%, or about 30% to 40%, or about 35% to 50%, or about 0.2% to 20%, or about 20% to 30%. For example, the formulation may have an amount of peptide of 0.1%, 0.2%, 0.5%, 0.8%, 1%, 1.5%, 2%, 5%, 10%, 25%, 40%, or 50%. In some instances, the formulation may have an amount of peptide up to about 0.1%, 0.2%, 0.5%, 0.8%, 1%, 1.5%, 2%, 5%, 10%, 25%, 40%, or 50%.

As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, for example, Remington: The Science and Practice of Pharmacy, 22d Edition, Loyd et al.eds., Pharmaceutical Press and Philadelphia College of Pharmacy at University of the Sciences (2012). Examples of physiologically acceptable carriers include buffers, such as phosphate buffers, citrate buffer, and buffers with other organic acids; antioxidants including ascorbic acid; low molecular weight (less than about 10 residues) polypeptides; proteins, such as serum albumin, gelatin, orimmunoglobulins; hydrophilic polymers, such as polyvinylpyrrolidone; amino acids such as glycine, glutamine, asparagine, arginine or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN® (ICI, Inc.; Bridgewater, New Jersey), polyethylene glycol (PEG), and PLURONICS™ (BASF; Florham Park, N.J.).

Compositions containing the peptides described herein or derivatives thereof suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

The described compositions may also contain adjuvants, such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like, may also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof is admixed with at least one inert customary excipient (or carrier), such as sodium citrate or dicalcium phosphate, or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active ingredient in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the peptides described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances and the like.

Besides such inert diluents, the composition can also include additional agents, such as wetting, emulsifying, suspending, sweetening, flavoring, or perfuming agents.

Suspensions, in addition to the active compounds, may contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances and the like.

Compositions of the peptides described herein or derivatives thereof for rectal administrations are optionally suppositories, which can be prepared by mixing the compounds with suitable non-irritating excipients or carriers, such as cocoa butter, polyethyleneglycol or a suppository wax, which are solid at ordinary temperatures but liquid at body temperature and, therefore, melt in the rectum or vaginal cavity and release the active component.

Dosage forms for topical administration of the peptides described herein or derivatives thereof include ointments, powders, sprays, and inhalants. The compounds described herein or derivatives thereof are admixed under sterile conditions with a physiologically acceptable carrier and any preservatives, buffers, or propellants as may be required. Ophthalmic formulations, ointments, powders, and solutions are also contemplated as being within the scope of the compositions.

The compositions can include one or more of the peptides described herein and a pharmaceutically acceptable carrier. As used herein, the term pharmaceutically acceptable salt refers to those salts of the peptides described herein or derivatives thereof that are, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the compounds described herein. The term salts refers to the relatively non-toxic, inorganic and organic acid addition salts of the compounds described herein. These salts can be prepared in situ during the isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See Barge et al., J. Pharm. Sci. 66:1-19 (1977), which is incorporated herein by reference in its entirety.)

Administration of the peptides and compositions described herein, or pharmaceutically acceptable salts thereof, can be carried out using pharmaceutically effective amounts for periods of time effective to treat a disease or condition. The effective amount of the peptides and compositions described herein, or pharmaceutically acceptable salts thereof as described herein, may be determined by one of ordinary skill in the art and includes exemplary dosage amounts for a mammal of from about 5 mg to 1 gram/kg of body weight of active peptide per day, which may be administered in a single dose or in the form of individual divided doses, such as from 2, 3, 4, 5, or 6 times per day. For example, the dosage amount can be from about 10 mg to 80 mg/kg of body weight of active peptide per day, about 400 mg to about 700 mg/kg of body weight of active compound per day, about 200 mg to about 800 mg/kg of body weight of active compound per day, about 500 mg to about 1 g/kg of body weight of active compound per day, about 100 mg to about 300 mg/kg of body weight of active compound per day, or about 800 mg to about 1000 mg/kg of body weight of active compound per day. In some aspects, the dosage amount can be up to about 100 mg/kg of body weight of active compound per day, about 200 mg/kg of body weight of active compound per day, about 400 mg/kg of body weight of active compound per day, about 600 mg/kg of body weight of active compound per day, about 800 mg/kg of body weight of active compound per day, or about 1000 mg/kg of body weight of active compound per day. As discussed in Section IV, depending on the disease or condition of the subject, a pharmaceutically effective amount of the peptide analogs, or salts thereof, may result in a different therapeutic effect.

Those of skill in the art will understand that the specific dose level and frequency of dosage for any particular subject may be varied and will depend upon a variety of factors, including the activity of the specific peptide analog employed; the metabolic stability and length of action of that compound; the species, age, body weight, general health, sex and diet of the subject; the mode and time of administration; rate of excretion; drug combination; the nature of the disease or condition experienced by the subject, and severity of the particular disease or condition. The precise dose to be employed in the formulation will also depend on the route of administration, and the seriousness of the disease or disorder, and should be decided according to the judgment of the practitioner and each subject's circumstances. Effective doses can be extrapolated from dose-response curves derived from in vitro or animal model test systems. Further, depending on the route of administration, one of skill in the art would know how to determine doses that result in a plasma concentration for a desired level of response in the cells, tissues and/or organs of a subject.

### IV. Methods of Use

Provided herein are methods of using the described Ang-(1-7) peptide analogs to treat various diseases and conditions. These diseases and conditions include, but are not limited to, a cancer, a cardiovascular disease or condition, a hypertension condition, a fibrotic condition, a metabolic condition, and inflammatory condition, an eye condition, a mental health condition, or a pain condition. Generally, the methods involve administering to a subject with a disease or condition an effective amount of one or more of the peptides or pharmaceutical compositions described herein, or a pharmaceutically acceptable salt or prodrug thereof. An effective amount, when used to describe an amount of the peptide analogs administered in provided methods, refers to the amount of the peptides that achieves the desired pharmacological effect or other biological effect. In some instances, the peptide analogs may be useful to treat diseases and disorders in which Ang-(1-7) may provide a benefit but has limited utility for therapeutic use because of its poor stability within the body.

As used herein, subject means both mammals and non-mammals. Mammals include, for example, humans; non-human primates, such as apes and monkeys; cattle; horses; sheep; rats; dogs; cats; mice; pigs; and goats. Non-mammals include, for example, fish, amphibians, reptiles, and birds. The peptides and compositions described herein, or pharmaceutically acceptable salts thereof, are useful for treating diseases and conditions in humans, including, without limitation, pediatric and geriatric populations, and in animals, such as for veterinary applications.

As used herein, the terms prevent, preventing, and prevention of a disease or disorder refer to an action, for example, administration of a composition or therapeutic agent, that occurs before or at about the same time a subject begins to show one or more symptoms of the disease or disorder, which inhibits or delays onset or severity of one or more symptoms of the disease or disorder.

As used herein the terms treatment, treat, or treating refer to a method of reducing one or more symptoms of a disease or condition. Thus in the disclosed method, treatment can refer to a 10%, 20%, 30%, 40%, 50%, 60%, 70%, 80%, 90%, or 100% reduction in the severity of one or more symptoms of the disease or condition. For example, a method for treating a disease is considered to be a treatment if there is a 10% reduction in one or more symptoms or signs (for example, size of the tumor or rate of tumor growth) of the disease in a subject as compared to a control. As used herein, control refers to the untreated condition (for example, the tumor cells not treated with the compounds and compositions described herein). Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. As used herein, references to decreasing, reducing, or inhibiting include a change of 10%, 20%, 30%, 40%, 50%, 60%, 70%, 80%, 90% or greater as compared to a control level. Such terms can include, but do not necessarily include, complete elimination.

The peptide analogs described herein are useful for both prophylactic and therapeutic methods of treatment. For prophylactic use, a pharmaceutically effective amount of the peptides and compositions, or pharmaceutically acceptable salts thereof, as described herein are administered to a subject prior to onset (that is before obvious signs of the diseases), during early onset (such as upon initial signs and symptoms of the disease), or after the development of the disease. Prophylactic administration can occur for several days to years prior to the manifestation of symptoms of disease. Therapeutic treatment involves administering to a subject a pharmaceutically effective amount of the compounds and compositions or pharmaceutically acceptable salts thereof as described herein after disease or condition is diagnosed.

In one aspect, provided herein are methods to treat or ameliorate cancer in a subject. Also provided are methods to prevent or reduce the likelihood of cancer occurring in a subject. Also provided are methods to prevent or reduce the likelihood of metastasis occurring in a subject. Also provided are methods of inhibiting cancer cell growth or proliferation in a subject, methods of inhibiting angiogenesis in a tissue, and methods of inhibiting fibrosis in a tissue. In some instances, the method of treating cancer in a subject includes administering to the subject an effective amount of an Ang-(1-7) peptide analog or pharmaceutical composition containing such a peptide. In some instances, administering the peptide analog or pharmaceutical composition inhibits cancer cell growth or proliferation in the subject. In some instances, the cancer comprises cells that express the angiotensin (1-7) receptor mas. In some instances, administering the peptide analog or pharmaceutical composition prevents or reduces the likelihood of tumor growth, metastasis, or both tumor growth and metastasis. In some instances, administering the peptide or composition inhibits at least one of cancer cell growth or proliferation, angiogenesis, inflammation, or fibrosis. In some instances, administering the peptide or composition inhibits cell growth or proliferation of endothelial cells (in vivo, in vitro). In some instances, administering the peptide or composition inhibits collagen formation.

Optionally, the cancer is prostate cancer, bladder cancer, bone cancer, brain cancer, breast cancer, colon cancer, cervical cancer, endometrial cancer, fallopian tube cancer, gastrointestinal cancer, genitourinary cancer, head and neck cancer, hepatocellular carcinoma, leukemia, lung cancer, lymphoma, melanoma, liver cancer, ovarian cancer, pancreatic cancer, peritoneal cancer, prostate cancer, renal cancer, sarcoma, skin cancer, or testicular cancer. In some instances, the cancer expresses the Ang-(1-7) receptor mas. In some cases, the mas receptor is overexpressed in the cancer. In one example, the cancer is lung cancer. In another example, the cancer is breast cancer. In another example, the cancer is glioblastoma. In another example, the cancer is prostate cancer. In another example, the cancer is sarcoma. In another example, the cancer is hepatocellular carcinoma.

Without being held to any particular theory, the Ang-(1-7) peptide analogs may have biological activity similar or improved as compared to the native Ang-(1-7) in treating or ameliorating cancer. For example, Ang-(1-7) reduces the growth of lung cancer (Gallagher and Tallant, Carcinogenesis 25:2015-52 (2003), Menon et al., Cancer Res. 15:2809-15 (2007), and Soto-Pantoj a et al., Mol. Canc. Ther. 8:1676-83 (2009)), breast cancer (Cook et al., Cancer Res. 70:8319-28 (2010)), glioblastoma (Moore et al., Free Radic. Biol. Med. 65:1060-8 (2013)), prostate cancer (Krishnan et al., The Prostate 73:60-70 (2013) and Krishnan et al., The Prostate 73:71-82 (2013)), and sarcoma (Petty et al., Clin. Cancer Res. 15:7398-404 (2009)) by decreasing cancer cell proliferation, reducing angiogenesis, attenuating inflammation, and decreasing fibrosis. In another example, Ang-(1-7) suppresses hepatocellular carcinoma growth and angiogenesis (Liu et al., Mol. Med. 21:626-36 (2015)). In another example, Ang-(1-7) reduces angiogenesis in lung cancer (Soto-Pantoja et al., Mol. Canc. Ther. 8:1676-83 (2009)). In some instances, Ang-(1-7) inhibits fibrosis in tissue such as, for example, reducing breast cancer fibrosis (Cook et al., Cancer Res. 70:8319-28 (2010)). In another example, Ang-(1-7) reduces metastasis (Krishnan et al., The Prostate 73:71-82 (2013)).

In some instances, the Ang-(1-7) analogs of this disclosure may inhibit cancer cell proliferation in vitro. In some instances, the cancer cells may be breast cancer cells or sarcoma cells. For example, analogs Ang-(1-7)-ACCA⁵, Ang-(1-7)-ACCA⁶, and Ang-(1-7)-ACCA³ may inhibit in vitro proliferation of 4T1 murine triple negative breast cancer cells and HT-1080 human sarcoma cells over the course of 3 days incubation, as shown in FIG. 4A and FIG. 4B. In some instances, the analogs may inhibit cancer cell proliferation about 20 to 30 percent as compared to untreated controls. In some instances, the provided analogs may inhibit cancer cell proliferation about the same extent as Ang-(1-7) but without need for repeated daily administration. In some instances, different types of cancer cells may be more or less sensitive to treatment with the Ang-(1-7) analogs. For example, all three analogs may inhibit cell proliferation similarly to each other and to Ang-(1-7) in breast cancer cells (specifically, 4T1 murine triple negative breast cancer cells) at both relatively low (100 nM) and relatively high (1 μM) concentrations, as shown in FIG. 4A. In another example, Ang-(1-7)-ACCA⁵ and Ang-(1-7) -ACCA³ may inhibit cell proliferation similarly to each other and two to three times more than Ang-(1-7) in sarcoma cells, as shown in FIG. 4B. In one example, HT-1080 human sarcoma cells were inhibited two to three times more by 1 μM Ang-(1-7)-ACCA⁵ and Ang-(1-7)-ACCA³ as compared to Ang-(1-7). In some instances, proliferation of sarcoma cancer cells may be reduced about 80% by 1 μM Ang-(1-7)-ACCA⁵ and Ang-(1-7)-ACCA³, as compared to untreated controls.

The methods of treating or preventing cancer in a subject can further comprise administering to the subject a therapeutic agent, radiation therapy, or a combination thereof. Thus, the provided compositions and methods can include one or more additional agents. The one or more additional agents and the peptides described herein, or pharmaceutically acceptable salts or prodrugs thereof, can be administered in any order, including concomitant, simultaneous, or sequential administration. Sequential administration can be administration in a temporally spaced order of up to several days apart. The methods can also include more than a single administration of the one or more additional agents and/or the compounds described herein or pharmaceutically acceptable salts or prodrugs thereof. The administration of the one or more additional agents and the compounds described herein or pharmaceutically acceptable salts or prodrugs thereof can be by the same or different routes and concurrently or sequentially.

Additional therapeutic agents include, but are not limited to, chemotherapeutic agents. A chemotherapeutic agent is a compound or composition effective in inhibiting or arresting the growth of an abnormally growing cell. Thus, such an agent may be used therapeutically to treat cancer as well as other diseases marked by abnormal cell growth. Illustrative examples of chemotherapeutic compounds include, but are not limited to, bexarotene, gefitinib, erlotinib, gemcitabine, paclitaxel, docetaxel, topotecan, irinotecan, temozolomide, carmustine, vinorelbine, capecitabine, leucovorin, oxaliplatin, bevacizumab, cetuximab, panitumumab, bortezomib, oblimersen, hexamethylmelamine, ifosfamide, CPT-11, deflunomide, cycloheximide, dicarbazine, asparaginase, mitotant, vinblastine sulfate, carboplatin, colchicine, etoposide, melphalan, 6-mercaptopurine, teniposide, vinblastine, antibiotic derivatives (including anthracyclines such as doxorubicin, liposomal doxorubicin, and diethylstilbestrol doxorubicin, bleomycin, daunorubicin, and dactinomycin); antiandrogens (such as enzalutamide, flutamide, nilutamide, bicalutamide, and ARN-509); antiestrogens (such as tamoxifen); antimetabolites (such as fluorouracil (FU), 5-FU, methotrexate, floxuridine, interferon alpha-2B, glutamic acid, plicamycin, mercaptopurine, and 6-thioguanine); cytotoxic agents (such as carmustine, BCNU, lomustine, CCNU, cytosine arabinoside, cyclophosphamide, estramustine, hydroxyurea, procarbazine, mitomycin, busulfan, cisplatin, vincristine and vincristine sulfate); hormones (such as medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (such as mephalen, chlorambucil, mechlorethamine (nitrogen mustard) and thiotepa); steroids (such as bethamethasone sodium phosphate); Akt inhibitors; glucocorticoid receptor inhibitors (such as beclometasone, betamethasone, budesonide, ciclesonide, flunisolide, fluticasone, mifepristone, mometasone, and triamcinolone); and survival factor inhibitors (such as inhibitors of neurotrophins, cytokines, epidermal growth factor (EGF), platelet-derived growth factor (PDGF), fibroblast growth factor (FGF), insulin-like growth factor (IGF), heparin-binding epidermal growth factor (HB-EGF), vascular endothelial growth factor (VEGF), pigment epithelium-derived factor (PEDF), schwannoma-derived growth factor (SDGF), hepatocyte growth factor (HGF), transforming growth factor-a (TGF-a), transforming growth factor-β (TGF-β), bone morphogenetic proteins (such as BMP1-BMP15), growth differentiation factor-9 (GDF-9), granulocyte-colony stimulating factor (G-CSF), granulocyte-macrophage colony stimulating factor (GM-CSF), myostatin (GDF-8), erythropoietin (EPO), and thrombopoietin (TPO)).

Optionally, the one or more additional agents can include antibodies. Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)2, Fab′ and the like. Antibodies may also be single-chain antibodies, chimeric antibodies, humanized antibodies or any other antibody derivative known to one of skill in the art that retains binding activity that is specific for a particular binding site. In addition, aggregates, polymers and conjugates of immunoglobulins or their fragments can be used where appropriate so long as binding affinity for a particular binding site is maintained. Exemplary antibodies include trastuzumab, alemtuzumab, ibritumomab, blinatumomab, bevacizumab, and cetuximab.

Optionally, the one or more additional agent can include cancer vaccines, such as, for example, sipuleucel-T (PROVENGE®, manufactured by Dendreon), which was approved in 2010 by the U.S. Federal and Drug Administration for use in some men with metastatic prostate cancer.

Any of the aforementioned additional agents can be used in any combination with the compositions described herein. Combinations are administered either concomitantly (such as an admixture), separately but simultaneously (such as via separate intravenous lines into the same subject), or sequentially (such as one of the compounds or agents is given first followed by the second). Thus, the term combination is used to refer to concomitant, simultaneous, or sequential administration of two or more agents.

The peptides described herein are also useful in stimulating Ang-(1-7) receptor mas activity in a cell. The methods of stimulating mas receptor activity in a cell include contacting the cell with an effective amount of one or more of the peptides or compositions as described herein. Optionally, the contacting is performed in vivo, such as, for example, wherein the cell is in a subject. Optionally, the contacting is performed in vitro.

In one aspect, provided herein are methods to treat or ameliorate cardiovascular disease, or a cardiovascular condition, in a subject.

In one instance, the methods may be used to treat, ameliorate, or prevent cardiac toxicity that arises from cancer treatment. Exemplary cardiac toxicities include cardiomyopathy, myocarditis, pericarditis, acute coronary syndromes, and congestive heart failure. Such toxicities may arise from targeted chemotherapeutic drugs such as, for example, anthracyclins; targeted therapeutics such as, for example, monoclonal antibodies and tyrosine kinase inhibitors; or radiation therapy. The peptide analogs of this disclosure may be administered to the subject either at the same time as the cancer treatment, before the cancer treatment, or after the cancer treatment. In some instances, the peptide analogs may be administered at the same time that the subject is being treated with a chemotherapeutic drug. In some instances, the peptide analogs may be administered before a subject receives radiation therapy. Administering the peptide analogs of the disclosure to a subject receiving, or who has received or will receive, cancer therapy may prevent or reduce the severity of cardiac damage that may be caused by the cancer therapy. In some instances, the method of preventing or reducing cardiac toxicity in a subject includes administering to a subject with cancer an effective amount of an Ang-(1-7) peptide analog, or pharmaceutical composition containing such a peptide, wherein the subject is being treating with a cancer therapy, will be treated with a cancer therapy, or has been treated with a cancer therapy. In some instances, the cancer therapy includes at least one of radiation therapy, a targeted chemotherapeutic drug, or a targeted therapeutic. Without being held to any particular theory, the Ang-(1-7) peptide analogs may have biological activity similar or improved as compared to the native Ang-(1-7) in treating, ameliorating, or preventing cardiac toxicity resulting from radiation therapy or treatment with a targeted chemotherapeutic drug as shown by Willey et al., Radiotherapy and Oncology, in press (2016) and Rahimi et al., Proceedings of the 106th Annual Meeting of the American Association for Cancer Research; Abstract 4489 (2015), respectively.

In one instance, the analogs may be used to treat, ameliorate, or prevent atherosclerosis. Atherosclerosis is a cardiovascular disease characterized by the deposition of plaque in the lumen of arteries. The plaque is composed of fats, cholesterol and calcium and is invaded by vascular smooth muscle cells, neutrophils and macrophages. As the plaque becomes larger and hardens, the arteries narrow, limiting the supply of blood to tissues. The plaque may also occlude the artery, leading to stroke or myocardial infarction. The plaque may also become unstable, release into the blood stream, and occlude smaller arterioles in the brain, heart, kidney or the periphery, leading to stroke, myocardial infarction, renal ischemia, or peripheral arterial disease, respectively. The provided methods may be used to treat a subject with atherosclerosis to reduce the severity of the disease symptoms. In some instances, the method of treating a subject diagnosed with atherosclerosis includes administering to the subject an effective amount of an Ang-(1-7) peptide analog or pharmaceutical composition containing such a peptide. In some instances, administering the disclosed peptide analogs may reduce or eliminate the symptoms of the disease. In some instances, administering the disclosed peptide analogs may prevent worsening of the symptoms of the disease. In some instances, administering the disclosed peptide analogs may reduce the amount of plaque formation in the subject. In some instances, administering the disclosed peptide analogs may stabilize established plaque formed within the subject. In some instances, administering the disclosed peptide analogs may prevent or reduce the likelihood of stroke or heart attack in a subject with established atherosclerosis. Without being held to any particular theory, the Ang-(1-7) peptide analogs may have biological activity similar or improved as compared to the native Ang-(1-7) in treating or ameliorating atherosclerosis. For example, Ang-(1-7) reduced plaque formation in mice lacking a protein that is essential for fat catabolism that were fed a high fat diet by reducing the growth and migration of vascular smooth muscle cells which infiltrate the plaque (Yang et al., Arteriosler. Thromb. Vasc. Biol. 33:1978-85(2013)). In another example, Ang-(1-7) increased the stability of established plaques, to prevent their release into the blood stream in a model of atherogenesis and plaque vulnuerability, by increasing the vascular production of collagen and reducing the migration of neutrophils and macrophages into the plaque (Fraga-Silva et al. Thromb. Haemost. 111:736-747 (2014)).

In another instance, the peptide analogs may be used to treat, ameliorate, or prevent thrombosis. Thrombosis is characterized by the formation of a thrombus, a blood clot inside a blood vessel, which can reduce or obstruct the flow of blood through the circulatory system. Thrombolytic reduction of blood flow can cause tissue hypoxia and more extreme obstruction can result in anoxia (the complete loss of oxygen), resulting in infarction. Thrombosis is an underlying pathology for a number of cardiovascular diseases, including myocardial infarction, stroke, and venous thromboembolism. In some instances, the method of treating a subject diagnosed with thrombosis, or at risk of thrombosis, includes administering to the subject an effective amount of an Ang-(1-7) peptide analog or pharmaceutical composition containing such a peptide. Without being held to any particular theory, the Ang-(1-7) peptide analogs may have biological activity similar or improved as compared to the native Ang-(1-7) in treating or ameliorating thrombosis. For example, pre-treatment with Ang-(1-7) significantly reduced thrombus formation in the abdominal vena cava of spontaneous hypertensive rats through activation of the mas receptor (Fraga-Silva et al., Clinics 66:837-841 (2011)). In another example, thrombus formation was also reduced in bradykinin receptor knockout mice with a decrease in nitric oxide and prostacyclin following Ang-(1-7) administration (Fang et al., Blood 121:3023-3032 (2013)).

In one instance, analogs of Ang-(1-7) may be used to treat patients with thrombocytopenia. Thrombocytopenia is characterized by an abnormally low concentration of platelets (thrombocytes) in the blood. Platelets are blood cell fragments required for clot formation. Decreased platelet concentration can be caused by reduced platelet production in the bone marrow or increased breakdown of platelets in the bloodstream, spleen, or liver. A number of pathological conditions can cause a reduction in platelet number, including aplastic anemia, cancer of the bone marrow, disseminated intravascular coagulation, drug-induced nonimmune thrombocytopenia, drug-induced immune thrombocytopenia, hypersplenism, liver cirrhosis, folate deficiency, bone marrow infections, myelodysplastic syndrome, thrombotic thrombocytopenic purpura, and vitamin B12 deficiency. The provided methods may be used to treat a subject suffering from thrombocytopenia by increasing the number of circulating platelets. In some instances, the method of treating a subject diagnosed with thrombocytopenia includes administering to the subject an effective amount of an Ang-(1-7) peptide analog or pharmaceutical composition containing such a peptide. Without being held to any particular theory, the Ang-(1-7) peptide analogs may have biological activity similar or improved as compared to the native Ang-(1-7) in treating or ameliorating thrombocytopenia. For example, Ang-(1-7) reduced the frequency of grade 2-4 thrombocytopenia as compared to filgrastim in patients with newly diagnosed breast cancer (Phase I/II trials) (Rodgers et al., Cancer Chemother Pharmacol 57:559-568 (2006)). In another example, Ang-(1-7) reduced Grade 3-4 thrombocytopenia following gemcitabine and platinum-based chemotherapy in ovarian cancer patients (Phase I/II trials) (Pham et al., Cancer Chemother Pharmacol 71:965-972 (2013)).

In one aspect, provided herein are methods to treat or ameliorate a hypertension condition in a subject.

In one instance, the peptide analogs may be used in methods to treat or amerliorate arterial hypertension or the resulting end organ damage. Arterial hypertension is a medical condition in which the blood pressure in a subject's arteries is persistently elevated. Sustained arterial hypertension may cause organ damage (referred to as end organ damage). In some instances, hypertensive end organ damage may include any of vascular and hemorrhagic stroke, retinopathy, coronary heart disease/myocardial infarction and heart failure, proteinuria and renal failure and in the vasculature, atherosclerotic change including the development of stenoses and aneurysms. In some instances, end organ damage may occur when a subject is considered pre-hypertensive (that is the subject has elevated blood pressure but above the lower threshold for treatment with a blood pressure medication). The provided methods may be used to treat subject with arterial hypertension or subjects who are considered pre-hypertensive to prevent or reduce the severity of end organ damage caused by the subject's elevated blood pressure. In some instances, the method of preventing or reducing blood pressure-induced end-organ damage in a subject includes administering to a subject having an elevated arterial blood pressure an effective amount of an Ang-(1-7) peptide analog or pharmaceutical composition containing such a peptide. In some instances, the subject has arterial hypertension. In some instances, the subject has elevated arterial blood pressure but has not been diagnosed with arterial hypertension. In some instances, the peptide analog or pharmaceutical composition may reduce blood pressure by decreasing the endothelial production of nitric oxide to increase vasodilation. In some instances, administering the peptide or composition reduces the elevated arterial blood pressure, reduces or prevents blood pressure-induced end-organ damage, or both.

Without being held to any particular theory, the Ang-(1-7) peptide analogs may have biological activity similar or improved as compared to the native Ang-(1-7) in treating, ameliorating, or preventing arterial hypertension or the resulting blood pressure-induced end organ damage. For example, Ang-(1-7) increases the production of nitric oxide to cause vasodilation in various vascular beds (Brosnihan et al., Hypertension, 27:523-8 (1996) and Osei et al., Eur. J. Pharmacol. 30:35-4 (1993)). The Ang-(1-7)-mediated reduction in end organ damage may include a decrease in hypertrophy, fibrosis or inflammation in the heart, the kidney or the blood vessels as Ang-(1-7) has been shown to reduce cardiac hypertrophy and fibrosis in various models of hypertension and in the heart/cardiac cells (McCollum et al., Am. J. Physiol. 302:H801-10 (2012), McCollum et al., Peptides 34:380-8 (2012), and Grobe et al., Am. J. Physiol. Heart Circ. Physiol. 290:H2417-23 (2006)), in the kidney (Benter et al., Am. J. Physiol. Heart Circ. Physiol. 290:H684-91 (2006)), and in the vasculature/vascular cells (Freeman et al., Hypertension 28:104-8 (1996) and Carver et al., Microcirculation 22:19-27 (2015)).

In another instance, the peptide analogs may be used in methods to treat, ameliorate, or prevent pulmonary hypertension. Pulmonary hypertension is a medical condition in which the blood pressure in a subject's lung vasculature, including any or all of the pulmonary artery, pulmonary vein, or pulmonary capillaries, is persistently elevated. In some instances, pulmonary hypertension causes shortness of breath, dizziness, fainting, leg swelling, and other symptoms, including, in severe cases, heart failure. In some instances, the peptide analogs of this disclosure are administered to a subject without also administering to the subject any other therapeutic composition. In some instances, the peptide analogs may be administered in conjunction with a therapeutic composition. Exemplary therapeutic compositions include prostaglandins, endothelin receptor agonists, phosphodiesterase type 5 inhibitors, and activators of soluble guanylate cyclase. The peptide analogs may be administered to the subject either at the same time as the additional therapeutic composition, before the additional therapeutic composition, or after the additional therapeutic composition. In some instances, the peptide analogs may be administered at the same time that the subject is being treated with an additional therapeutic composition. In some instances, the peptide analogs may be administered before a subject is administered any additional therapeutic composition. Administering the peptide analogs of the disclosure to a subject who has pulmonary hypertension may prevent or reduce the severity of the disease. In some instances, the method of preventing or reducing pulmonary hypertension in a subject includes administering to a subject having an elevated pulmonary blood pressure an effective amount of an Ang-(1-7) peptide analog or pharmaceutical composition containing such a peptide. In some instances, administering the peptide analog or pharmaceutical composition may reduce fibrosis and inflammation in the heart or decrease fibrosis and hypertrophy in the pulmonary vasculature of the subject. In some instances, administering the peptide or composition prevents or reduces pulmonary blood pressure or symptoms relating thereto. Without being held to any particular theory, the Ang-(1-7) peptide analogs may have biological activity similar or improved as compared to the native Ang-(1-7) in treating or ameliorating pulmonary hypertension. For example, Ang-(1-7) prevented the development of pulmonary hypertension and the associated cardiopulmonary pathology in rats administered monocrataline and significantly reversed these pathologies in rodents with established pulmonary hypertension, to improve heart function and reduce pulmonary vessel fibrosis (Shenoy et al., Hypertension 64:1248-1259 (2010.

In one instance, the Ang-(1-7) analogs may be used to treat erectile dysfunction. Erectile dysfunction is characterized by the inability to maintain a penile erection sufficient for sexual intercourse. The release of neurotransmitters, particularly nitric oxide, cause relaxation of smooth muscle cells in cavernosal arterioles and sinuses, resulting in blood flow to the penis and erection. Erectile dysfunction is caused by failure of the neuronal response, an increase in vascular tone, and/or contractility of the smooth muscle within the corpus cavernosum and penile arteries. Chronic vascular changes can impair the arterial response to vasodilators, which can lead to permanent erectile dysfunction. In some instances, the method of treating a patient suffering from erectile dysfunction may include administering to the subject an effective amount of an Ang-(1-7) peptide analog or pharmaceutical composition containing such a peptide. In some instances, administering the peptide or composition may cause relaxation of smooth muscle cells in cavernosal arterioles and sinuses, resulting in blood flow to the penis and erection. Without being held to any particular theory, the Ang-(1-7) peptide analogs may have biological activity similar or improved as compared to the native Ang-(1-7) in treating or ameliorating erectile dysfunction. For example, treatment with Ang-(1-7) caused relaxation of the corpus cavernosum and release of nitric oxide as well as restored erectile function in hypertensive rats (da Costa Goncalves et al., Am. J. Physiol. Heart Circ. Physiol. 293:H2588-H2596 (2007)).

In one aspect, provided herein are methods to treat or ameliorate a fibrotic-related condition in a subject. Exemplary fibrotic-related conditions include radiation-induced fibrosis and endometriosis.

In one instance, the peptide analogs may be used in methods to treat or ameliorate radiation-induced fibrosis. Radiation-induced fibrosis involves the abnormal production of the protein, fibrin, which accumulates in and damages the radiated tissue. Any tissue within the radiation field can be affected including nerves, muscles, blood vessels, bones, tendons, skin, ligaments, gastrointestinal and genitourinary tracts, heart, lungs, or other organs, depending on the treatment site. Radiation-induced fibrosis may cause both cosmetic and functional impairment, which can lead to death or a significant deterioration in the quality of life. The development of radiation-induced fibrosis is influenced by multiple factors, including the radiation dose and volume, fractionation schedule, previous or concurrent treatments, genetic susceptibility, and comorbidities such as diabetes mellitus, heart disease, and arthritis. The peptide analogs of this disclosure may be administered to the subject either at the same time as radiation therapy, before radiation therapy, or after radiation therapy. In some instances, the peptide analogs may be administered at the same time that the subject is being treated with a radiation therapy. In some instances, the peptide analogs may be administered before a subject receives radiation therapy. In some instances, the peptide analogs may be administered before a subject receives radiation therapy. Administering the peptide analogs of the disclosure to a subject receiving, or who has received or will receive, radiation therapy may prevent or reduce the severity of fibrotic damage or inflammation that may be caused by the radiation therapy. In some instances, the method of preventing or reducing radiation-induced fibrosis in a subject includes administering to a subject with cancer who is being treated with radiation therapy, will be treated with radiation therapy, or has been treated with radiation therapy an effective amount of an Ang-(1-7) peptide analog or pharmaceutical composition containing such a peptide. In some instances, administering the peptide or composition prevents or reduces radiation-induced fibrosis. Without being held to any particular theory, the Ang-(1-7) peptide analogs may have biological activity similar or improved as compared to the native Ang-(1-7) in treating or ameliorating radiation-induced fibrosis. For example, Ang-(1-7) reduced fibrosis in the hind limb of mice treated with radiation, in association with a reduction in inflammatory mediators, to prevent the loss of muscle function (Willey et al., Radiotherapy and Oncology, in press (2016)).

In another instance, the peptide analogs may be used in methods to treat or ameliorate endometriosis. Endometriosis refers to a condition in which the uterine-lining tissue grows outside the uterus, usually on the surfaces of organs in the pelvic and abdominal areas (and, rarely, in other areas as well). Endometriosis most commonly involves one or more of a subject's ovaries, uterus, bladder, bowel, or the tissue lining the pelvis. Symptoms may include abdominal pain, heavy menstrual periods, and infertility. Typical treatment options include pain relievers, hormones, and surgery. The provided methods may be used to treat subject with endometriosis to reduce the severity of the condition. In some instances, the method of treating a subject diagnosed with endometriosis includes administering to the subject an effective amount of an Ang-(1-7) peptide analog or pharmaceutical composition containing such a peptide. In some instances, the method prevents or reduces endometrial tissue growth outside the subject's uterus. In some instances, administering the disclosed peptide analogs may slow the growth of the endometrial tissue, reduce fibrosis and tissue scarring associated with the disease, decrease inflammation, or a combination thereof. In some instances, administering the disclosed peptide analogs may cause regression of the endometrial tissue that has grown outside of the subject's uterus. Without being held to any particular theory, the Ang-(1-7) peptide analogs may have biological activity similar or improved as compared to the native Ang-(1-7) in treating or ameliorating endometriosis.

In one aspect, provided herein are methods to treat or ameliorate degenerative neurological conditions in a subject. In one instance, the peptide analogs may be used in methods to treat or ameliorate Alzheimer's disease (AD). AD is a chronic neurodegenerative disease that is associated with the build-up of extracellular amyloid “plaques” in variable shapes and sizes in the brain. The plaques primarily comprise amyloid beta protein fibrils. In addition, AD is characterized by the presence of numerous neurofibrillary “tangles”, comprising paired helical filaments of amyloid beta protein that abnormally accumulate in the neuronal cytoplasm. The other major type of lesion found with AD is the accumulation of amyloid beta protein in the walls of blood vessels, both within the brain parenchyma and in the walls of meningeal vessels that lie outside the brain. Amyloid beta protein is produced when the amyloid protein precursor (APP) undergoes successive proteolysis by beta- and gamma-secretase. Accumulating evidence implicates amyloid, and more specifically, the formation, deposition, accumulation and/or persistence of amyloid beta plaques and tangles, as a major causative factor of AD pathogenesis. The provided methods may be used to treat a subject with AD to reduce the severity of the disease symptoms. In some instances, the method of treating a subject diagnosed with AD includes administering to the subject an effective amount of an Ang-(1-7) peptide analog or pharmaceutical composition containing such a peptide. In some instances, administering the disclosed peptide analogs may reduce or eliminate the symptoms of the disease. In some instances, administering the disclosed peptide analogs may prevent worsening of the symptoms of the disease. In some instances, administering the disclosed peptide analogs may reduce the amount of amyloid plaques, amyloid tangles, or both, in the subject. In some instances, administering the disclosed peptide analogs may reduce the amount of amyloid beta protein in the walls of blood vessels of the subject. In some instances, administering the peptide or composition prevents or reduces muscle fibrosis or improves muscle function. Without being held to any particular theory, the Ang-(1-7) peptide analogs may have biological activity similar or improved as compared to the native Ang-(1-7) in treating or ameliorating Alzheimer's disease. For example, Ang-(1-7) significantly prevented cognitive deficits in rats subjected to chronic cerebral hypo-perfusion, a characteristic of Alzheimer's disease, by altering nitric oxide production and reducing the proliferation of astrocytes (Xie et al., Brain Research 1573:44-53 (2014). The provided methods may be used to treat a patient suffering from AD to reduce the severity of the disease symptoms and increase cerebral perfusion.

In another aspect, the peptide analogs may be used in methods to treat or ameliorate muscular dystrophy in the subject. The muscular dystrophies (MD) are a group of more than 30 genetic diseases that are characterized by progressive muscle weakness and degeneration of the skeletal muscles that control movement. Different forms of MD are seen in infancy or childhood, while others may not appear until middle age or later. The provided methods may be used to treat a subject with MD to reduce the severity of the disease symptoms. In some instances, the method of treating a subject diagnosed with MD includes administering to the subject an effective amount of an Ang-(1-7) peptide analog or pharmaceutical composition containing such a peptide. Administering the analog or composition may reduce muscle fibrosis and delay the loss of muscle use. In some instances, administering the disclosed peptide analogs may reduce the symptoms of the disease. In some instances, administering the disclosed peptide analogs may delay worsening of the symptoms of the disease. Without being held to any particular theory, the Ang-(1-7) peptide analogs may have biological activity similar or improved as compared to the native Ang-(1-7) in treating or ameliorating muscular dystrophy. For example, Ang-(1-7) decreased muscle fibrosis and improved muscle function in a mouse model of MD (Acuna et al., Hum. Mol. Genet. 23:1237-49 (2014).

In one aspect, provided herein are methods to treat or ameliorate a metabolic condition in a subject. Exemplary metabolic condition include diabetes-induced end organ damage and metabolic syndrome.

In one aspect, the peptide analogs may be used in methods to treat or ameliorate diabetes-induced end organ damage in the subject. Diabetes mellitus is a metabolic disorder in which a subject's ability to moderate blood glucose levels in response to insulin is lost. Complications from diabetes include any of increased risk of cardiovascular disease, neuropathy, nephropathy, retinopathy, foot damage, skin conditions, hearing impairment, and Alzheimer's disease. The provided methods may be used to treat a subject with diabetes to reduce the severity of the disease symptoms. In some instances, administering the disclosed peptide analogs may reduce or eliminate the symptoms of the disease. In some instances, administering the disclosed peptide analogs may prevent worsening of the symptoms of the disease. In some instances, administering the disclosed peptide analogs may prevent further cellular damage within the subject. In some instances, administering the disclosed peptide analogs may result in improved glucose metabolism for subject's with type 2 diabetes. In some instances, the method of treating a subject diagnosed with diabetes includes administering to the subject an effective amount of an Ang-(1-7) peptide analog or pharmaceutical composition containing such a peptide. In some instances, the subject may be pre-diabetic. In some instances, the subject may be glucose insensitive. Without being held to any particular theory, the Ang-(1-7) peptide analogs may have biological activity similar or improved as compared to the native Ang-(1-7) in treating or ameliorating diabetes-induced end organ damage. For example, Ang-(1-7) reduces oxidative stress, fibrosis and inflammation, but increases nitric oxide levels within diabetic tissues (Alzayadneh and Chappell, Cell Signaling,12:3026, (2014); Giani et al,, Am. J. Physiol.:Renal, 302:F1605 (2012); Zhang et al., Kidney Int. 87:359 (2015)).

In one instance, the peptide analogs may be used to treat or ameliorate metabolic syndrome. Metabolic syndrome is a group of pathologies that act in a synergistic manner to promote cardiovascular disease and an overall decline in health. The metabolic syndrome is characterized by hypertension, obesity, hyperlipidemia and insulin insensitivity (or hyperglycemia). In some instances, the method of treating a subject diagnosed with metabolic syndrome includes administering to the subject an effective amount of an Ang-(1-7) peptide analog or pharmaceutical composition containing such a peptide. In some instances, the administration of the Ang-(1-7) analogs may reduce or reverse elevated blood pressure, weight gain, or both, in the subject. Administration of the analog may in some instances may restore insulin sensitivity and normalize blood glucose levels. In some instances, the Ang-(1-7) analogs may lower or restore normal blood lipid levels. Additionally, the administration of Ang-(1-7) analogs in conjunction with moderate exercise may result in a greater improvement in the metabolic syndrome than exercise alone. Without being held to any particular theory, the Ang-(1-7) peptide analogs may have biological activity similar or improved as compared to the native Ang-(1-7) in treating or ameliorating metabolic syndrome. For example, Ang-(1-7) reduced insulin and insulin-mediated signaling mechanisms, decreased inflammation through a reduction in proinflammatory cytokines, and reduced body weight and fat deposition (Giani et al, Am. J Physiol: Endocrin., 296:E262-71 (2009); Feltenberger et al, Hypertension 62:324-30 (2013); Santos et al, Peptides 46:47-52 (2013); Andrade et al, Peptides 55:56-62 (2014)).

In another aspect, provided herein are methods to treat or ameliorate an inflammatory condition in a subject. Exemplary inflammatory conditions include acute pancreatitis, rheumatoid arthritis, acute respiratory distress syndrome, asthma, cirrhosis, and uveitis.

In one instance, the analogs may be used to treat acute pancreatitis. Acute pancreatitis involves sudden inflammation of the pancreas which may lead to a severe, life-threating illness. Patients with acute pancreatitis may suffer from internal bleeding within the organ resulting in tissue damage, infection or cyst formation. Acute pancreatitis may also result in damage to other organs including the heart, the lungs and the kidneys, further increasing morbidity and mortality. The provided methods may be used to treat a subject with acute pancreatitis to reduce the severity of the disease symptoms. In some instances, the method of treating a subject diagnosed with acute pancreatitis includes administering to the subject an effective amount of an Ang-(1-7) peptide analog or pharmaceutical composition containing such a peptide. In some instances, administering the disclosed peptide analogs may reduce or eliminate the symptoms of the disease. In some instances, administering the disclosed peptide analogs may prevent worsening of the symptoms of the disease. In some instances, administering the disclosed peptide analogs may reduce inflammation within the pancreas and, in some instances, other organs as well. Without being held to any particular theory, the Ang-(1-7) peptide analogs may have biological activity similar or improved as compared to the native Ang-(1-7) in treating or ameliorating acute pancreatitis. For example, in pancreatic cells treated with cerulean to simulate acute pancreatitis, treatment with Ang-(1-7) increased anti-inflammatory cytokines and decreased inflammatory cytokines to reduce damage to pancreatic cells by increasing nitric oxide/nitric oxide signaling pathways (Wang et al., Pancreas 44:266-272 (2015)).

In another instance, the analogs may be used to treat rheumatoid arthritis. Rheumatoid arthritis is an inflammatory disease of the joints. It is an autoimmune disease where the immune system mistakenly attacks the joints, causing chronic joint inflammation and pain. Although inflammation of the tissue around the joints and inflammatory arthritis are characteristic features of rheumatoid arthritis, rheumatoid arthritis can also cause inflammation and injury in other organs in the body, resulting in a systemic illness. The provided methods may be used to treat a patient suffering from rheumatoid arthritis to reduce the severity of the disease symptoms and the associated pain. In some instances, the method of treating a subject diagnosed with rheumatoid arthritis includes administering to the subject an effective amount of an Ang-(1-7) peptide analog or pharmaceutical composition containing such a peptide. In some instances, administration of the peptides analogs may reduce inflammation. In some cases, the peptides analogs may reduce migration of inflammatory neutrophils to a subject's joints. In some instances, the peptides analogs may reduce the production of inflammatory cytokines. Without being held to any particular theory, the Ang-(1-7) peptide analogs may have biological activity similar or improved as compared to the native Ang-(1-7) in treating or ameliorating rheumatoid arthritis. For example, Ang-(1-7) reduced inflammation in two different rodent models of arthritis by reducing inflammation, the infiltration of inflammatory neutrophils, and the production of inflammatory cytokines (Da Silveria et al., J. Immunology 185:5569-76 (2010).

In one instance, the analogs may be used to treat acute respiratory distress syndrome. Acute respiratory distress syndrome is a lung condition characterized by acute onset of respiratory distress and low blood oxygen levels. Patients present with diffuse alveolar and capillary membrane damage, fluid accumulation, and heightened inflammatory responses. Acute respiratory distress syndrome can be caused by trauma, sepsis, drug overdose, environmental toxins, massive transfusion of blood products, acute pancreatitis, or aspiration. In some instances, the method of treating a patient diagnosed with acute respiratory distress syndrome may include administering to the subject an effective amount of an Ang-(1-7) peptide analog or pharmaceutical composition containing such a peptide. In some instances, administration of the peptide analog or composition may reduce inflammation and associated damage in the lungs of the subject. Without being held to any particular theory, the Ang-(1-7) peptide analogs may have biological activity similar or improved as compared to the native Ang-(1-7) in treating or ameliorating acute respiratory distress syndrome. For example, treatment with Ang-(1-7) significantly reduced lung injury and inflammation as well as improved oxygenation in a rat model of acute respiratory distress syndrome (Zambelli et al., Intensive Care Med. Exp. 3:44 (2105).

In one instance, the analogs may be used to treat asthma. Asthma is a chronic, reversible, inflammatory lung disorder that results in obstruction and thickening of the airways. Asthma is characterized by an enhanced inflammatory response, mucosal edema, pulmonary fibrosis, increased secretion by the mucosa, smooth muscle hypertrophy and hyperplasia, basement membrane degradation, reduced epithelial cell integrity, smooth muscle cell contractility, loss of cartilage and angiogenesis. In some instances, the method of treating a subject diagnosed with asthma includes administering to the subject an effective amount of an Ang-(1-7) peptide analog or pharmaceutical composition containing such a peptide. Without being held to any particular theory, the Ang-(1-7) peptide analogs may have biological activity similar or improved as compared to the native Ang-(1-7) in treating or ameliorating asthma. For example, Ang-(1-7) attenuated allergic inflammation through activation of the mas receptor in a mouse model of asthma, by reducing neutrophil infiltration, the production of cytokines, and perivascular and peribronchial inflammation, fibrosis and hyperplasia (El-Hashim et al., British Journal of Pharm. 166:1964-76 (2012)).

In another instance, the analogs may be used to treat cirrhosis. Cirrhosis is a chronic liver disease liver characterized by increased inflammation, tissue fibrosis, and liver cell destruction. Cirrhosis is commonly caused by chronic and excessive alcohol intake, hepatitis, and non-alcoholic fatty liver disease. Enhanced inflammatory responses result in stellate cell activation, which promotes fibrosis through production of myofibroblasts and secretion of TGF-β. Fibrotic tissue blocks portal blood flow, leading to reduced tissue oxygenation and loss of function. In some instances, cirrhosis may be considered a fibrotic-related condition. In some instances, the method of treating a subject diagnosed with cirrhosis includes administering to the subject an effective amount of an Ang-(1-7) peptide analog or pharmaceutical composition containing such a peptide. In some instances, administering the peptide analogs may reduce the severity of the disease symptoms. In some instances, administration of the peptides analogs may reduce inflammation. In some instances, administering the peptide analogs may improve circulation in the liver. In some instances, administering the peptide analogs may reduce fibrosis in the liver. Without being held to any particular theory, the Ang-(1-7) peptide analogs may have biological activity similar or improved as compared to the native Ang-(1-7) in treating or ameliorating cirrhosis. For example, Ang-(1-7) increased splanchnic circulation and portal blood flow while decreasing hepatic resistance in a model of liver cirrhosis (Grace et al., Am J Gastrointest Liver Physiol 304:G99-G108 (2013)).

In one instance, the analogs may be used to treat patients with uveitis. Uveitis is an inflammatory eye disorder of the pigmented middle layer of tissue in the eye wall (uvea) that lies between the inner retina and the outer fibrous layer. The uvea is highly vascularized and provides blood supply to many parts of the eye. Uveitis can be caused by viral infection, systemic inflammatory diseases, eye injury, allergic inflammation, autoimmune disorders and systemic diseases, such as Lyme disease, sarcoidosis, and juvenile rheumatoid arthritis. In some instances, the method of treating a subject diagnosed with uveitis includes administering to the subject an effective amount of an Ang-(1-7) peptide analog or pharmaceutical composition containing such a peptide. The provided methods may be used to treat a patient suffering from uveitis to reduce the severity of the disease symptoms. In some instances, administering the peptide analogs may reduce inflammation in the eye. Without being held to any particular theory, the Ang-(1-7) peptide analogs may have biological activity similar or improved as compared to the native Ang-(1-7) in treating or ameliorating uveitis. For example, oral feeding of bioencapsulased Ang-(1-7) significantly reduced cellular infiltration and retinal vasculitis in a mouse model of autoimmune uveoretinitis (Shil et al., Molecular Therapy 22:2069-2082 (2014)).

In one instance, the Ang-(1-7) analogs may be used to treat glaucoma. Glaucoma is characterized by insufficient drainage of aqueous humor which can lead to increased intraocular pressure within the eyeball. Over time, the elevated pressure can damage the optic nerve, resulting in gradual loss of sight. Without treatment, glaucoma can cause total permanent blindness within a few years. The provided methods may be used to treat a patient suffering from uveitis to reduce the severity of the disease symptoms. In some instances, the method of treating a subject diagnosed with glaucoma includes administering to the subject an effective amount of an Ang-(1-7) peptide analog or pharmaceutical composition containing such a peptide. In some instances, administering the peptide analogs may reduce ocular pressure and/or inflammation associated with elevated ocular pressure. Without being held to any particular theory, the Ang-(1-7) peptide analogs may have biological activity similar or improved as compared to the native Ang-(1-7) in treating or ameliorating glaucoma. For example, intravitreal administration of Ang-(1-7) significantly reduced intraocular pressure through activation of the mas receptor (Vaajanen et al., Invest. Ophthalmol. Vis. Sci., 49:2557-2562 (2008)).

In some instances, the analogs may be used to treat an eye condition such as uveitis or glaucoma.

In one aspect, the analogs may be used to treat a mental health condition such as emotional or mental stress. Emotional or mental stress is a state of strain or tension resulting from potential or actual adverse, demanding, or even threatening circumstances. This condition can result in increased sympathetic outflow that elevates blood pressure and heart rate. Increased risk of cardiovascular disorders is an adverse consequence of chronic emotional stress. The provided methods may be used to treat a patient suffering from emotional or mental stress, to reduce the severity of the disease symptoms. In some instances, the method of treating a subject diagnosed with emotional or mental stress includes administering to the subject an effective amount of an Ang-(1-7) peptide analog or pharmaceutical composition containing such a peptide. In some instances, administering the peptide analogs may reduce elevated blood pressure, elevated heart rate, or both in a subject. Without being held to any particular theory, the Ang-(1-7) peptide analogs may have biological activity similar or improved as compared to the native Ang-(1-7) in treating or ameliorating emotional or mental stress. For example, microinjection of Ang-(1-7) into the central nervous system or into the basolateral amygdala of emotionally stressed rats caused a significant reduction in tachycardia and the pressor response produced by air jet stress (Lima et al., Am. J. Physiol. Heart Circ. Physiol. 305:H1057-H1067 (2013); Oscar et al., Brain Research 1594:183-189 (2015)).

In another aspect, the Ang-(1-7) analogs may be used to treat a pain condition such as nociceptive pain. Nociceptive pain is caused by stimulation of peripheral nerve fibers, called nociceptors, that recognize and react to a tissue damaging stimulus, such as pressure, tearing, shearing, extreme temperatures, activating substances released by other cells, or environmental chemicals, and send pain signals through the nervous system. Pain of damaged body tissue as well as pain caused by pathological conditions such as cancer, diabetic peripheral neuropathy, postherpetic neuralgia, and arthritis, are considered nociceptive in origin. In some instances, the method of treating a subject diagnosed with nociceptive pain includes administering to the subject an effective amount of an Ang-(1-7) peptide analog or pharmaceutical composition containing such a peptide. In some instances, administering the peptide analogs reduces nociceptive pain felt by the subject. Without being held to any particular theory, the Ang-(1-7) peptide analogs may have biological activity similar or improved as compared to the native Ang-(1-7) in treating or ameliorating nociceptive pain. For example, Ang-(1-7) induced an antinociceptive effect independent of an opioid pathway (Costa et al., Pharmacology 89:137-144 (2012)) but with an activation of the L-arginine/nitric acid/cGMP and K⁺ATP pathways (Costa et al., Nitric Oxide 37:11-16 (2014)).

### V. Kits

Also provided herein are kits for treating or preventing cancer in a subject. In one aspect, the kits are for treating cancer in a subject. In another aspect, the kits are for use in preventing cancer in a subject. A kit can include any of the peptides or compositions described herein, or pharmaceutically acceptable salts thereof. For example, a kit can include one or more peptides Formula I or pharmaceutically acceptable salts thereof. A kit can further include one or more additional agents, such as a chemotherapeutic agent. A kit can include an oral formulation of any of the peptides or compositions described herein. A kit can additionally include directions for use of the kit (such as instructions for treating a subject), a container, a means for administering the compounds or compositions, and/or a carrier. Kits can include single doses or multiple doses (such as, for example, in a blister pack or a multi-dose volume vial). Kits can include can include means for administration (such as a delivery device like a syringe, a nebulizer, or an inhaler), or the like.

Non-limiting embodiments include:

Embodiment 1. A peptide comprising the formula X¹—X²—X³—X⁴—X⁵—X⁶—X⁷, wherein:
X¹ is aspartic acid, N-methyl aspartic acid, alanine, or N-methyl alanine;
X² is arginine, N-methyl arginine, or cis-3-(aminomethyl)cyclobutanecarboxylic acid (ACCA);
X³ is valine, N-methyl valine, alanine, N-methyl alanine, or ACCA;
X⁴ is tyrosine, N-methyl tyrosine, phenylalanine, N-methyl phenylalanine, alanine, N-methyl alanine, or ACCA;
X⁵ is isoleucine, N-methyl isoleucine, alanine, N-methyl alanine, leucine, N-methyl leucine, or ACCA;
X⁶ is histidine, N-methyl histidine, alanine, N-methyl alanine, or ACCA; and
X⁷ is proline, N-methyl proline, alanine, or N-methyl alanine;
   wherein at least one of X², X³, X⁴, X⁵, and X⁶ is ACCA.

Embodiment 2. The peptide of embodiment 1, wherein at least two of X², X³, X⁴, X⁵, and X⁶ are ACCA.

Embodiment 3. The peptide of embodiment 1, wherein the peptide comprises an amino acid sequence as set forth in SEQ ID Nos. 2, 2, 3, 4, 5, 6, 7, 8, 9, 10, 11, 12, 13, 14, 15, or 16.

Embodiment 4. The peptide of embodiment 1, wherein the peptide comprises the formula N¹—X¹—X²—X³—X⁴—X⁵—X⁶—X⁷—C¹—Y¹, wherein:
N¹ is norleucine (Nle), leucine (L), alanine (A), norvaline (Nva), azidohomoalanine (Aha), or 2-Aminobutyric acid (Abu);
C¹ is lysine (K), ornithine (Orn), 2,3-diaminopropionic acid (Dap), 2,4-diaminobutyric acid (Dab), or N-methyl lysine (NMe-K); and
Y¹ is absent, a single amino acid, or two amino acids.

Embodiment 5. The peptide of embodiment 4, wherein the peptide is a cyclic peptide.

Embodiment 6. The peptide of embodiment 4 or 5, wherein N¹ or X¹ is connected to C¹ via a lactam bridge thereby cyclizing the peptide.

Embodiment 7. The peptide of any one of embodiments 1-6, wherein the peptide has a longer half-life than angiotensin (1-7) in biological conditions.

Embodiment 8. A pharmaceutical composition comprising a pharmaceutically effective amount of the peptide of any of embodiments 1-7 and a pharmaceutically acceptable carrier.

Embodiment 9. The pharmaceutical composition of embodiment 8, wherein the concentration of the peptide is in the range of 30 mg/ml to 100 mg/ml.

Embodiment 10. The pharmaceutical composition of embodiment 8 or 9, wherein the amount of the peptide is in the range of 5 mg to 1 gram.

Embodiment 11. A method of treating a subject with a disease or condition, the method comprising administering to a subject a pharmaceutically effective amount of the peptide of any one of embodiments 1-7 or the composition of any one of embodiments 8-10.

Embodiment 12. The method of embodiment 11, wherein the disease or condition is at least one of a cancer, a cardiovascular disease or condition, a hypertension condition, a fibrotic condition, a metabolic condition, and inflammatory condition, an eye condition, a mental health condition, or a pain condition.

Embodiment 13. The method of embodiment 11 or 12, wherein the subject has at least one of cancer, atherosclerosis, thrombosis, thrombocytopenia, elevated arterial blood pressure, pulmonary hypertension, thrombosis, erectile dysfunction, endometriosis, Alzheimer's disease, muscular dystrophy, diabetes, metabolic syndrome, acute pancreatitis, rheumatoid arthritis, acute respiratory distress syndrome, asthma, cirrhosis, uveitis, glaucoma, emotional and mental distress, or nocicieptive pain.

Embodiment 14. The method of any one of embodiments 11-13, wherein the subject has been diagnosed with cancer.

Embodiment 15. The method of any one of embodiments 11-14, wherein administering the peptide or composition inhibits at least one of cancer cell growth or proliferation, angiogenesis, inflammation, or fibrosis.

Embodiment 16. The method of any one of embodiments 11-15, wherein the subject has been diagnosed with cancer and is being treating with a cancer therapy, will be treated with a cancer therapy, or has been treated with a cancer therapy.

Embodiment 17. The method of any one of embodiments 11-16, wherein administering the peptide or composition prevents or reduces cardiac toxicity.

Embodiment 18. The method of any one of embodiments 11-17, wherein the cancer therapy comprises at least one of radiation therapy, a targeted chemotherapeutic drug, or a targeted therapeutic.

Embodiment 19. The method of embodiment 11, wherein the subject has been diagnosed with atherosclerosis.

Embodiment 20. The method of embodiment 11, wherein the subject has been diagnosed with thrombosis.

Embodiment 21. The method of embodiment 11, wherein the subject has been diagnosed with thrombocytopenia.

Embodiment 22. The method of embodiment 11, wherein the subject has an elevated arterial blood pressure.

Embodiment 23. The method of embodiment 22, wherein administering the peptide or composition reduces the elevated arterial blood pressure, reduces or prevents blood pressure-induced end-organ damage, or both.

Embodiment 24. The method of embodiment 22 or 23, wherein the subject has elevated arterial blood pressure but has not been diagnosed with arterial hypertension.

Embodiment 25. The method of embodiment 11, wherein the subject has pulmonary hypertension.

Embodiment 26. The method of embodiment 11, wherein the subject has been diagnosed with thrombosis.

Embodiment 27. The method of embodiment 11, wherein the subject has been diagnosed with erectile dysfunction.

Embodiment 28. The method of embodiment 11, wherein the subject has cancer and is being treated with radiation therapy, will be treated with radiation therapy, or has been treated with radiation therapy.

Embodiment 29. The method of embodiment 28, wherein administering the peptide or composition prevents or reduces radiation-induced fibrosis.

Embodiment 30. The method of embodiment 11, wherein the subject has endometriosis.

Embodiment 31. The method of embodiment 11, wherein the subject has Alzheimer's disease.

Embodiment 32. The method of embodiment 11, wherein the subject has muscular dystrophy.

Embodiment 33. The method of embodiment 11, wherein the subject has diabetes.

Embodiment 34. The method of embodiment 32, wherein administering the peptide or composition prevents or reduces diabetes-induced end organ damage.

Embodiment 35. The method of embodiment 11, wherein the subject has metabolic syndrome.

Embodiment 36. The method of embodiment 11, wherein the subject has acute pancreatitis.

Embodiment 37. The method of embodiment 11, wherein the subject has rheumatoid arthritis.

Embodiment 38. The method of embodiment 11, wherein the subject has acute respiratory distress syndrome.

Embodiment 39. The method of embodiment 11, wherein the subject has asthma.

Embodiment 40. The method of embodiment 11, wherein the subject has cirrhosis.

Embodiment 41. The method of embodiment 11, wherein the subject has uveitis.

Embodiment 42. The method of embodiment 11, wherein the subject has glaucoma.

Embodiment 43. The method of embodiment 11, wherein the subject has emotional and mental distress.

Embodiment 44. The method of embodiment 11, wherein the subject has nocicieptive pain.

Embodiment 45. A method of inhibiting angiogenesis in a subject, the method comprising administering to a subject diagnosed with a cancer an effective amount of the peptide of any one of embodiments 1-7 or the composition of any one of embodiments 8-10.

Embodiment 46. A method of inhibiting fibrosis in a subject, the method comprising administering to a subject an effective amount of the peptide of any one of embodiments 1-7 or the composition of any one of embodiments 8-10.

Embodiment 47. A method of inhibiting inflammation in a subject, the method comprising administering to a subject an effective amount of the peptide of any one of embodiments 1-7 or the composition of any one of embodiments 8-10.

Embodiment 48. A method of stimulating mas receptor in a cell, the method comprising administering subject diagnosed with a cancer an effective amount of the peptide of any one of embodiments 1-7 or the composition of any one of embodiments 8-10.

### EXAMPLES

### EXAMPLE 1: Synthesis of ACCA

Reagents and solvents were purchased from commercial suppliers and used as received unless noted otherwise.

The non-natural amino acid ACCA was synthesized according to the method depicted in Scheme 1. Procedures for each step of the synthesis are provided below and are also described in O'Reilly et al., Amino Acids 44:511-518 (2013), the content of which is incorporated herein by reference in its entirety. ACCA was synthesized as an HCl salt.

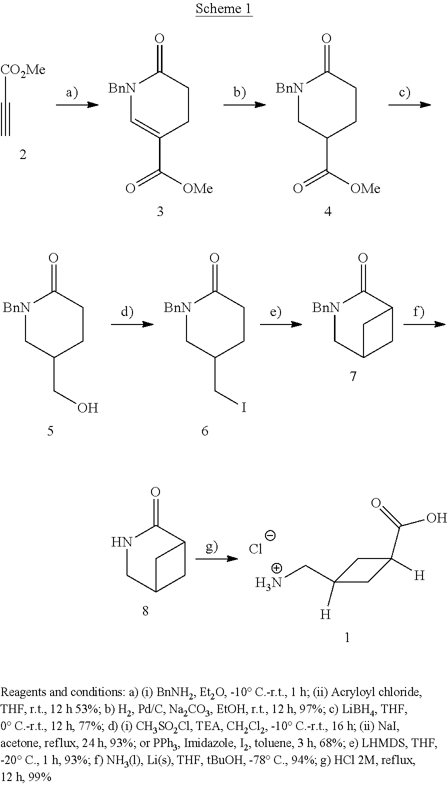

Compounds 3 and 4 were prepared as described in Cook et al., JOC 59 (13):3575-3584 (1994), the content of which is incorporated herein in its entirety.

Preparation of (±)1-Benzyl-5-(hydroxymethyl)piperidin-2-one (5). (±) Compound 4 (5.23 g, 23.85 mmol) was dissolved in anhydrous THF (35 mL) and cooled to 0° C. LiBH₄ (1.04 g, 47.70 mmol) was added and the solution was allowed to warm to room temperature and stirred overnight. The reaction was quenched at 0° C. with water (20 mL) and then with 10% HCl. The organic layer was separated and the aqueous layer was extracted with EtOAc (4×20 mL). The organic layers were combined, dried over MgSO₄ and concentrated in vacuo. The crude oil was purified by silica gel column chromatography (cyclohexane/EtOAc, 60:40) to give (±) Compound 5 (4.03 g, 77% yield) as a clear oil. (Rf=0.11, cyclohexane/EtOAc, 50:50). δH (600 MHz; CDCl₃) 7.33-7.18 (5H, m, PhH), 4.59 (1H, d, J=14.6, PhCH₂), 4.53 (1H, d, J=14.6, PhCH₂), 3.53 (1H, dd, J=10.7 and 5.6, CH₂OH), 3.44 (1H, dd, J=10.7 and 7.2, CH₂OH), 3.30 (1H, ddd, J=12.2, 5.2 and 1.5, NCH₂), 2.99 (1H, dd, J=12.2 and 10.1, NCH₂), 2.92 (1H, br s, OH), 2.51 (1H, ddd, J=17.8, 5.8 and 3.4, C═OCH₂), 2.39 (ddd, J=17.8, 11.3 and 6.5, C═OCH₂), 2.04-1.95 (1H, m, OHCH₂CH), 1.89-1.82 (1H, m, C═OCH₂CH₂), 1.54-1.46 (1H, m, C═OCH₂CH₂); δC (151 MHz; CDCl₃) 170.0, 137.0, 128.5, 127.9, 127.3, 64.3, 50.3, 49.8, 36.4, 31.1, 23.8; m/z (ES) 220.1329 (M+H⁺C₁₃H₁₈NO₂ requires 220.1338).

Preparation of (±) 1-Benzyl-5-(iodomethyl)piperidin-2-one (6). (±) Compound 5 (3.92 g, 13.18 mmol) was dissolved in anhydrous CH₂Cl₂ (25 mL). The solution was cooled to—10° C. and TEA (2.0 g,19.77 mmol) was added, followed by the slow addition of CH₃SO₂Cl (1.81 g, 15.19 mmol). The solution was allowed to warm slowly to room temperature and stirred overnight. The organic layer was concentrated in vacuo and dissolved in acetone (50 mL) and NaI (3.98 g, 26.58 mmol) was added to the stirring solution. The mixture was stirred under reflux for 24 hours. The organic solvent was removed in vacuo and H₂O (40 mL) was added along with EtOAc (50 mL), the organic layer was separated and the aqueous layer was extracted with EtOAc (5×20 mL). The organic layers were combined, washed with brine, dried over MgSO₄ and concentrated in vacuo. The crude oil was purified by silica gel column chromatography (cyclohexane/EtOAc, 70:30) to give Compound 6 (3.36 g, 96% yield) as a yellow/orange oil. (Rf=0.12, cyclohexane/EtOAc, 50:50). δH (500 MHz; CDCl₃) 7.3-7.22 (5H, m, PhH), 4.63 (1H, d, J=14.7, PhCH₂), 4.54 (1H, d, J=14.7, PhCH₂), 3.34 (1H, m, NCH₂), 3.08 (2H, m, CH₂I), 2.97 (1H, dd, J=12.0 and 9.5, NCH₂), 2.56 (1H, ddd, J=17.7, 6.2 and 3.6, C═OCH₂), 2.45 (1H, ddd, J=17.7, 11.2 and 6.2, C═OCH₂), 2.06-1.95 (2H, m, C═OCH₂CH₂), 1.66-1.54 (1H, m, CHCH₂); δC (126 MHz; CDCl₃) 169.0, 136.8, 128.5, 128.0, 127.4, 52.3, 50.1, 35.9, 30.7, 27.7, 7.9; m/z (EI) 329.0273 (M+H⁺. C₁₃H₁₆INO requires 329.0277).

Preparation of 3-Benzyl-3-aza-bicyclo[3.1.1]heptan-2-one (7). (±) Compound 6 (3.2 g, 9.72 mmol) was dissolved in anhydrous THF (5 mL) and LHMDS (1M soln./THF, 9.72 mL) was added at −20° C. The solution was stirred for one hour before being quenched with H₂O (15 mL). EtOAc (20 mL) was added and the organic layer was separated. The aqueous layer was extracted with EtOAc (3×15 mL). The organic layers were combined, dried over MgSO₄ and concentrated in vacuo. The crude oil was filtered through a short silica column (cyclohexane/EtOAc, 2:1) to give Compound 7 (1.82 g, 93% yield) as a yellow/orange oil. (Rf=0.24, cyclohexane/EtOAc, 50:50). δH (500 MHz; CDCl₃) 7.36-7.22 (5H, m, PhH), 4.61 (2H, s, PhCH₂), 3.26 (2H, d, J=1.5, NCH₂), 2.84 (1H, q, J =5.7 Hz, C═OCH), 2.72-2.65 (1H, m, NCH₂CH), 2.38-2.28 (2H, m, C═OCH(CHH)2, 1.73-1.63 (2H, m, C═OCH(CHH)₂; δC (126 MHz; CDCl₃) 175.7, 137.4, 128.5, 128.0, 127.2, 50.0, 48.0, 41.1, 33.2, 30.9; m/z (ES) 202.1222 (M+H⁺ C₁₃H₁₆NO requires 202.1232).

Preparation of 3-aza-bicyclo[3.1.1]heptan-2-one (8). Compound 7 (1.5 g, 7.45 mmol) was dissolved in anhydrous THF/tBuOH, 10:1 (10 mL) and the mixture was added to a stirring liquid ammonia at −78° C. Metallic Li pellets were added slowly to the stirring solution until a constant blue/black colour was observed. The reaction was then quenched by the addition of solid ammonium chloride. After removal of the NH₃, EtOAc (30 mL) and H₂O (8 ml) were added. The organic layer was separated and the aqueous layer was extracted with EtOAc (3×15 mL). The organic layers were combined, dried over MgSO₄ and concentrated in vacuo. The crude oil was filtered through a short silica column (cyclohexane/EtOAc, 1:1) to give Compound 8 (800 mg, 94% yield) as a yellow oil. (Rf=0.1, cyclohexane/EtOAc, 50:50). δH (500 MHz; CD₃OD) 7.46-7.13 (1H, m, NH) 3.43 (2H, d, J=2.0, NCH₂), 2.81-2.71 (1H, m, NCH₂CH), 2.60 (1H, q, J=5.6 Hz, C═OCH), 2.48-2.37 (2H, m, C═OCH(CHH)₂, 1.70-1.62 (2H, m, C═OCH(CHH)₂). δC (126 MHz; CD₃OD) 181.5, 46.3, 42.0, 34.3, 31.4; m/z (EI+) 111.0684 (M+H⁺ C₆H₉NO requires 111.0684).

Preparation of cis-3-(Aminomethyl)cyclobutanecarboxylic acid (1·HCL). Compound 8 (220 mg, 1.98 mmol) was refluxed in 2M HCl (10 mL) overnight. The H₂O was removed in vacuo to give Compound 1 (328 mg, quantitative yield) as a beige solid with no further purification required. Mp: 169-170° C. δH (500 MHz; CD₃OD) 3.18-3.08 (1H, m, C═OCH), 2.96 (2H, d, J=7.3, NCH₂), 2.63-2.51 (1H, m, NCH₂CH), 2.46-2.37 (2H, m, C═OCH(CHH)₂, 2.02 (2H, m, C═OCH(CHH)₂; δC (126 MHz; CD₃OD) 176.8, 45.5, 34.9, 30.2, 29.9; m/z (ES⁺) 130.0863 (M+H⁺ C₆H₁₂NO₂ requires 130.0868).

### EXAMPLE 2: Synthesis of Ang-(1-7) Analogs

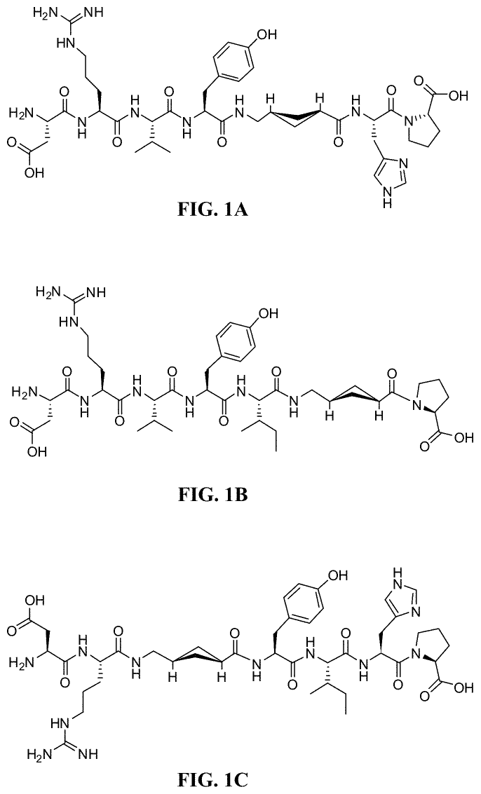

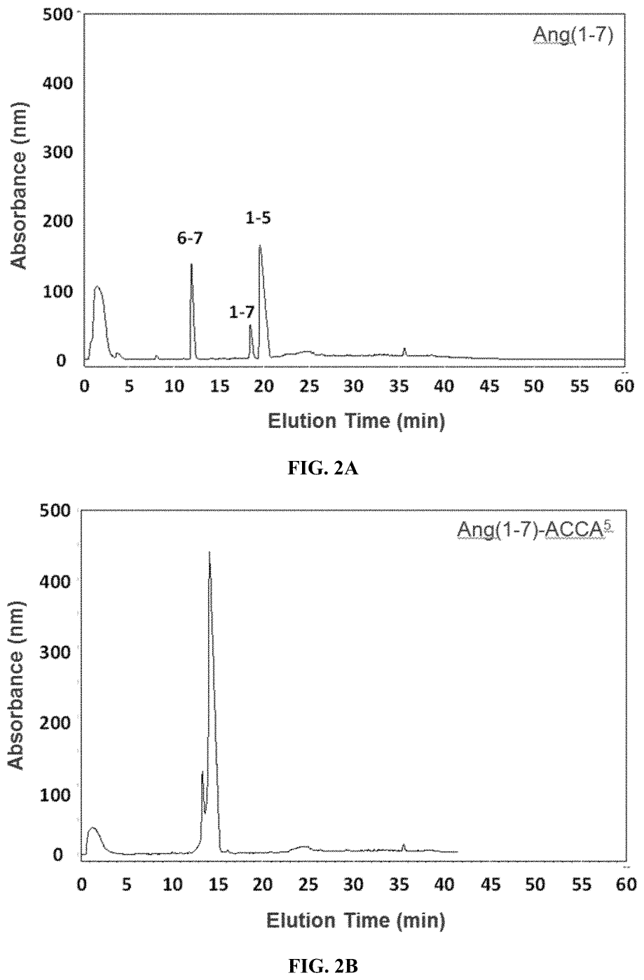

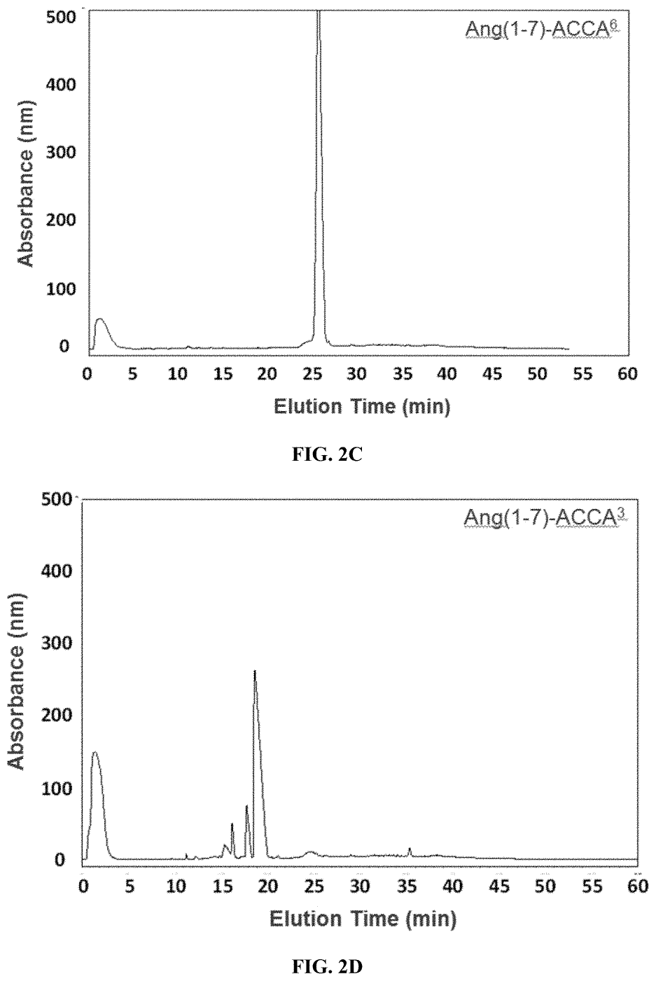

Exemplary Ang(1-7) analogs were synthesized by substituting ACCA at position 5 (Ang-(1-7)-ACCA⁵), position 6 (Ang-(1-7)-ACCA⁶), and position 3 (Ang-(1-7)-ACCA³) to give Compounds 9, 10 and 11, respectively, as shown in FIGS. 1A-1C.

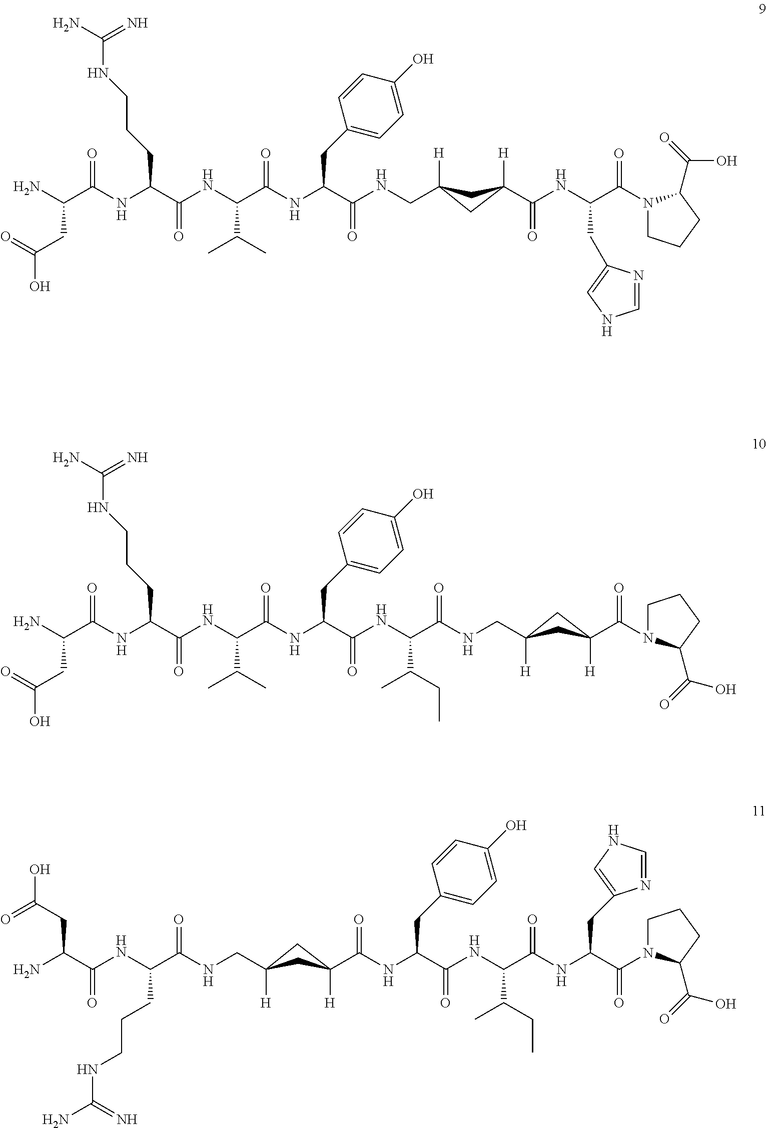

FMOC-protection of ACCA was carried out in order to facilitate its use in solid phase peptide synthesis (SPPS) using FMOC-OSu and aqueous sodium carbonate in dioxane (Scheme 2) yielding FMOC-ACCA 12 in 95% yield. The formation of Compound 12 was confirmed by X-ray crystallography.

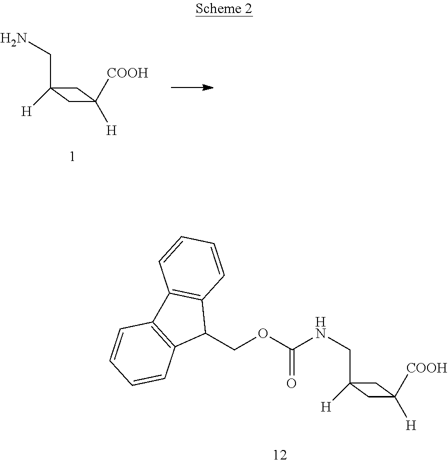 [SMILES: NC[C@H]1C[C@@H](C(=O)O)C1 ; O=C(NC[C@H]1C[C@@H](C(=O)O)C1)OCC1c2ccccc2-c2ccccc21]

Reagents and conditions: FMOC-OSu, 10% aq Na₂CO₃, dioxane, 0° C. - r.t., 1 h 45 min, 95%.

N-(9-Fluorenylmethoxycatbonyl)-cis-3-(aminomethyl) cyclobutane carboxylic acid (12). Compound 1 (ACCA) (1 eq, 0.78 mmol) was dissolved 10% (w/v) aq. Na₂CO₃ (2.5 eq, 1.95 mmol), 1,4- dioxane (5 mL) was added and the temperature was brought to 0° C. FMOC-OSu (1.5 eq, 1.17 mmol) in 1,4- dioxane (2.5 mL) was added and the mixture wad stirred for 45 min at 0° C. and for 1 h at room temperature. The reaction mixture was then washed with Et₂O (30 mL). The aqueous layer was acidified with 1 M HCL and extracted with EtOAc (4×25 mL). The combined organic layers were washed with brine, dried over Na₂SO₄, filtered and concentrated in vacuo. The crude material was purified by silica gel column chromatography /cyclohexane/EtOAc, from 95:5 to 40:60) and crystallized from acetone and petroleum sprits (b.p. 40-60° C.) to give the product (260 mg, 95%) as a white solid: Rf=0.36 (cyclohexane/EtOAc, 1:5); m.p. 134-135° C.; ¹H NMR (500 MHz, (CD₃)₂CO) δ=7.70 (m, 2 H, Ph), 7.54-7.53 (m, 2 H, Ph), 7.27-7.16 (m, 4H, Ph), 6.41 (s, 1 H, NH), 4.18 (d, J=7.2 Hz, 2 H, C═OOCH₂), 4.08-4.06 (m, 1 H, C═OOCH₂CH), 3.06-3.02 (m, 2 H, NCH₂), 2.91-2.83 (m, 1 H, CHC═OOH), 2.38-2.29 (m, 1 H, NCH₂CH), 2.15-2.09 (m, 2 H, C═OCH(CHH)₂), 1.88-1.82 (m, 2H, C═OCH(CHH)₂); ¹³C NMR (126 MHz, CD₃OD) δ=175.4, 156.4, 144.3 (2 C), 141.2(2 C,) 127.6(2 C), 127.0(2 C), 125.2(2 C), 119.8 (2 C), 65.9, 47.2, 45.7 33.5, 31.2, 28.5 (2 C); HRMS (ES) m/z=374.1377 [M+Na] C₂₁H₂₁NO₄Na requires 374.1368.

Peptide synthesis, purification and characterization. FMOC-protected amino acids, HATU ((1-[Bis(dimethylamino)methylene]-1H-1,2,3-triazolo[4,5-b]pyridinium 3-oxid hexafluorophosphate)), and proline pre-loaded 2CT resin were purchased from Iris Biotec. All other reagents and solvents were purchased from Sigma-Aldrich. Single coupling cycles (except for the amino acid coupling to the resin-bound proline, double coupling cycle) using a 10-fold excess of Fmoc-protected amino acid to resin substitution were carried out employing HATU/DIEA coupling chemistry in NMP solvent. The side-chain protecting groups were Trt for His, t-Bu for Tyr and Asp and Pbf for Arg. The synthesis was carried out on a 0.1 mmol scale using an automated peptide synthesizer (Applied Biosystems 433A). Peptide cleavage from the resin and side chain deprotection was achieved by gentle stirring in a mixture of 87.5% TFA, 5% H₂O, 5% thioanisole and 2.5% triisopropylsilane for 2.5 h at room temperature. The TFA and scavenger mixture was evaporated under a stream of nitrogen and the peptide was precipitated by the addition of cold ether. The suspension was centrifuged for 5 min at 8000 rpm, the supernatant decanted and the peptides were redissolved in ether. This was repeated three times. The peptides were then air dried, redissolved in double distilled water and lyophilized. Chromatographic analysis and purification were performed on a Shimadzu HPLC using Aeris Peptide columns (Phenomenex, 3.6 μ XB-C18, 100 mm×2.1 mm, 150 mm×4.6 mm, for the analytic and semi-preparative columns respectively). Mobile phases consisted of A: H₂O (0.1% phosphoric acid); B: 80% MeOH, 20% H₂O (0.1% phosphoric acid). A gradient program was used were 0-15% B in 20 min, to 35% B in 10 min, hold for 5 min, back to 0% B in 15 min, hold for 5 min with a flow rate of 0.35 mL/min (analytical and semi-preparative) and UV detection at 220 nm.

Final product characterization:

| Compound | Amino Acid Sequence | Characterization |
|---|---|---|
| Compound 9 | H-Asp-Arg-Val-Tyr- | white solid, yield = 64%, Rₜ = 14.5 |
| [Ang-(1-7)- | ACCA-His-Pro-OH | min; HRMS (ES) m/z = 897.4585 |
| ACCA⁵] | (SEQ ID NO: 5) | [M + H] C₄₁H₆₁N₁₂O₁₁ requires |
| | | 897.4583 |
| Compound 10 | H-Asp-Arg-Val-Tyr- | white solid, yield = 46%, Rₜ = 25.6 |
| [Ang-(1-7)- | Ile-ACCA-Pro-OH | min; HRMS (ES) m/z = 873.4811 |
| ACCA⁶] | (SEQ ID NO:6) | [M + H] C₄₁H₆₅N₁₀O₁₁ requires |
| | | 873.4834 |
| Compound 11 | H-Asp-Arg-ACCA-Tyr- | white solid, yield = 63%, Rₜ = 17.5 |
| [Ang-(1-7)- | Ile-His-Pro-OH | min; HRMS (ES) m/z = 911.4761 |
| ACCA³] | (SEQ ID NO: 3) | [M + H] C₄₂H₆₃N₁₂O₁₁ requires |
| | | 911.4739 |

### EXAMPLE 3: Degradation by ACE

Analogs with ACCA at position 5 (Ang-(1-7)-ACCA⁵), position 6 (Ang-(1-7)-ACCA⁶), and position 3 (Ang-(1-7)-ACCA³) were assessed to determine whether the analogs were degraded by ACE. Ang-(1-7) served as the control. Specifically, 100 μM of one of the three analogs or Ang-(1-7) was incubated with 1.0 μg of human ACE in reaction buffer (25 mM Hepes, 125 mM NaCl 10 μM ZnCl₂, pH 7.4) for 2 hours at 37° C. in a final volume of 100 μL. The resultant reaction products were separated by high pressure liquid chromatography (HPLC) on a Phenomenex Aeris Peptide C18 column using a Shimadzu Prominence system and detected by UV analysis at 220 nm. Ang-(1-7) was degraded to Ang-(1-5) and the dipeptide His-Pro (amino acids 6 and 7) (FIG. 2A), while Ang-(1-7)-ACCA⁵ and Ang-(1-7)-ACCA⁶ were resistant to metabolism by ACE (FIG. 2B and FIG. 2C, respectively). However, the Ang-(1-7)-ACCA³ was not resistant to ACE and was metabolized at a similar rate as the Ang-(1-7) (FIG. 2D). These results clearly demonstrate that the incorporation of the non-natural amino acid ACCA in place of isoleucine and histidine (Ang-(1-7)-ACCA⁵ and Ang-(1-7)-ACCA⁶), but not valine (Ang-(1-7)-ACCA³), prevents degradation of the Ang-(1-7) analogs by ACE. This suggests that the ACCA analogues will be more stable and have an enhanced half-life as compared to Ang-(1-7).

### EXAMPLE 4: Degradation by DPP III

The Ang-(1-7) and the analogs Ang-(1-7)-ACCA⁵, Ang-(1-7)-ACCA⁶, and Ang-(1-7) -ACCA³ were assessed to determine whether they were resistant to metabolism by the enzyme dipeptidyl peptidase III (DPP III). DPP III metabolizes Ang-(1-7) at different sites as compared to ACE (Chappell et al., Front Endocrinol (Lausanne) 4:201 (pp. 1-13) (2014)). The incubation conditions and detection methods for the metabolism by DPP III were identical to that used to assess degradation by ACE as described in Example 3. Human DPP III sequentially degraded Ang-(1-7) to Ang-(5-7) and the dipeptides Asp-Arg and Val-Tyr by (FIG. 3A). The Ang-(1-7)-ACCA⁵ and Ang-(1-7)-ACCA⁶ analogs were also metabolized by DPP III to a similar extent as Ang-(1-7) and, thus, do not appear to be resistant to the DPP III peptidase (FIG. 3B and FIG. 3C). However, the Ang-(1-7)-ACCA³ analog, having ACCA substitution of the valine residue, was completely resistant to DPP III and showed no evidence of metabolism (FIG. 3D). Overall, these studies demonstrate that the three ACCA analogs exhibit a difference in their resistance to metabolism by ACE and DPP III, and that the ACCA analogs will be more stable and have an enhanced half-life as compared to the Ang-(1-7).

### EXAMPLE 5: In Vitro Inhibition of Cancer Cell Growth

The ability of the analogs Ang-(1-7)-ACCA⁵, Ang-(1-7)-ACCA⁶, and Ang-(1-7)-ACCA³ to inhibit cell proliferation in cancer cells was compared to that of Ang-(1-7). Specifically, rapidly proliferating 4T1 murine triple negative breast cancer cells or HT-1080 human sarcoma cells, growing in 24 well cluster plates in DMEM with 1% fetal bovine serum, were incubated with 100 nM Ang-(1-7), added daily to replenish the degraded peptide, or either 100 nM or 1 μM of the analogs, for a total of 3 days of treatment. The total number of cells per well was counted using a Nexelcom Cellometer. Ang-(1-7) served as the control, and was added daily due to its rapid degradation in serum. In contrast, the analogs were added to the cell culture media at the beginning of the study and were not replenished daily. Breast cancer cells: n=3 in duplicate; sarcoma cells: n=2 in duplicate. Data analysis was by ANOVA with posthoc tests using Dunnett's Multiple Comparison Test.

Ang-(1-7) at 100 nM effectively reduced proliferation of the mouse triple negative breast cancer cells as compared to the untreated control cells (FIG. 4A). Similarly, the Ang-(1-7) -ACCA⁵ and Ang-(1-7)-ACCA⁶ analogs significantly attenuated growth of the 4T1 breast cancer cells at a concentration of 100 nM and all three analogs decreased proliferation of the cancer cells at 1 μM. The proliferation of human HT-1080 sarcoma cells was reduced about 80% by 1 μM of analogs Ang-(1-7)-ACCA⁵ and Ang-(1-7)-ACCA³, as compared to untreated controls (FIG. 4B). The efficacy of analogs to inhibit proliferation over the length of the study (after being only at the start of the experiment) demonstrates the enhanced stability of the modified peptides as compared to Ang-(1-7). These results suggest that the ACCA analogs of Ang-(1-7) may serve as effective chemotherapeutic agents through a reduction in cancer cell proliferation, similar to Ang-(1-7).

While aspects of the invention will now be described in connection with certain preferred embodiments in the following examples and with reference to the attached figures so that aspects thereof may be more fully understood and appreciated, it is not intended to limit the invention to these particular embodiments. On the contrary, this application is intended to cover all alternatives, modifications and equivalents as may be included within the scope of the invention as defined by the appended claims. Thus, the following examples, which include preferred embodiments, will serve to illustrate the practice of the described compositions, methods, and kits, it being understood that the particulars shown are by way of example and for purposes of illustrative discussion of preferred embodiments only, and are presented in the cause of providing what is believed to be useful and readily understood description of formulation procedures, as well as of the principles and conceptual aspects of the invention. It will be evident to those skilled in the art that the invention described herein may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

All printed patents and publications referred to in this application are hereby incorporated by reference herein in their entirety.

1. A peptide comprising the formula X¹—X²—X³—X⁴—X⁵—X⁶—X⁷, wherein:
X¹ is aspartic acid, N-methyl aspartic acid, alanine, or N-methyl alanine;
X² is arginine, N-methyl arginine, or cis-3-(aminomethyl)cyclobutanecarboxylic acid (ACCA);
X³ is valine, N-methyl valine, alanine, N-methyl alanine, or ACCA;
X⁴ is tyrosine, N-methyl tyrosine, phenylalanine, N-methyl phenylalanine, alanine, N-methyl alanine, or ACCA;
X⁵ is isoleucine, N-methyl isoleucine, alanine, N-methyl alanine, leucine, N-methyl leucine, or ACCA;
X⁶ is histidine, N-methyl histidine, alanine, N-methyl alanine, or ACCA; and
X⁷ is proline, N-methyl proline, alanine, or N-methyl alanine;
wherein at least one of X², X³, X⁴, X⁵, and X⁶ is ACCA.

2. The peptide of claim 1, wherein at least two of X², X³, X⁴, X⁵, and X⁶ are ACCA.

3. The peptide of claim 1, wherein the peptide comprises an amino acid sequence as set forth in SEQ ID Nos. 2, 2, 3, 4, 5, 6, 7, 8, 9, 10, 11, 12, 13, 14, 15, or 16.

4. The peptide of claim 1, wherein the peptide comprises the formula N¹—X¹—X²—X³—X⁴—X⁵—X⁶—X⁷—C¹—Y¹, wherein:
N¹ is norleucine (Nle), leucine (L), alanine (A), norvaline (Nva), azidohomoalanine (Aha), or 2-Aminobutyric acid (Abu);
C¹ is lysine (K), ornithine (Orn), 2,3-diaminopropionic acid (Dap), 2,4-diaminobutyric acid (Dab), or N-methyl lysine (NMe-K); and
Y¹ is absent, a single amino acid, or two amino acids.

5. The peptide of claim 1, wherein the peptide is a cyclic peptide.

6. The peptide of claim 5, wherein N¹ or X¹ is connected to C¹ via a lactam bridge thereby cyclizing the peptide.

7. The peptide of claim 1, wherein the peptide has a longer half-life than angiotensin (1-7) in biological conditions.

8. A pharmaceutical composition comprising a pharmaceutically effective amount of the peptide of claim 1 and a pharmaceutically acceptable carrier.

9. The pharmaceutical composition of claim 8, wherein the concentration of the peptide is in the range of 30 mg/ml to 100 mg/ml.

10. The pharmaceutical composition of claim 8, wherein the amount of the peptide is in the range of 5 mg to 1 gram.

11. A method of treating a subject with a disease or condition, the method comprising administering to a subject a pharmaceutically effective amount of the peptide of claim 1.

12. The method of claim 11, wherein the disease or condition is at least one of a cancer, a cardiovascular disease or condition, a hypertension condition, a fibrotic condition, a metabolic condition, and inflammatory condition, an eye condition, a mental health condition, or a pain condition.

13. The method of claim 11, wherein the subject has at least one of cancer, atherosclerosis, thrombosis, thrombocytopenia, elevated arterial blood pressure, pulmonary hypertension, thrombosis, erectile dysfunction, endometriosis, Alzheimer's disease, muscular dystrophy, diabetes, metabolic syndrome, acute pancreatitis, rheumatoid arthritis, acute respiratory distress syndrome, asthma, cirrhosis, uveitis, glaucoma, emotional and mental distress, or nocicieptive pain.

14. (canceled)

15. The method of claim 11, wherein administering the peptide or composition inhibits at least one of cancer cell growth or proliferation, angiogenesis, inflammation, or fibrosis.

16. The method of claim 11, wherein the subject has been diagnosed with cancer and is being treating with a cancer therapy, will be treated with a cancer therapy, or has been treated with a cancer therapy.

17. The method of claim 11, wherein administering the peptide or composition prevents or reduces cardiac toxicity.

18. (canceled)

19. (canceled)

20. (canceled)

21. (canceled)

22. (canceled)

23. (canceled)

24. (canceled)

25. (canceled)

26. A method of inhibiting angiogenesis in a subject, the method comprising administering to a subject diagnosed with a cancer an effective amount of the peptide of claim 1.

27. A method of inhibiting fibrosis in a subject, the method comprising administering to a subject an effective amount of the peptide of claim 1.

28. A method of inhibiting inflammation in a subject, the method comprising administering to a subject an effective amount of the peptide of claim 1.

29. A method of stimulating mas receptor in a cell, the method comprising contacting the cell with an effective amount of the peptide of claim 1.
